SEQUENCE LISTING (12 sequences)

SEQ ID NO: 1 - protein, 18 aa, Artificial sequence
  Exemplary recombinant PLK1 substrate peptide.
XXAXXXPLHSTXXXXXXX

SEQ ID NO: 2 - protein, 18 aa, Artificial sequence
  Exemplary recombinant PLK1 substrate peptide.
XXAFDPPLHSTXXXXXXX

SEQ ID NO: 3 - protein, 18 aa, Artificial sequence
  Exemplary recombinant PLK1 substrate peptide.
XXAFDPPLHSTAIXXXXX

SEQ ID NO: 4 - protein, 18 aa, Artificial sequence
  Exemplary recombinant PLK1 substrate peptide.
YEAFDPPLHSTAIYADEE

SEQ ID NO: 5 - protein, 18 aa, Artificial sequence
  Exemplary recombinant PLK1 substrate peptide.
FEAFDPPLHSTAIFADEE

SEQ ID NO: 6 - protein, 87 aa, Artificial sequence
  Exemplary recombinant PLK1 substrate peptide.
XEAFDPPLHSTAIXADEEGGPGGXEAFDPPLHSTAIXADEEGGPGGXEAFDPPLHSTAIXADEEGGPGGXEAFDPPLHSTAIXADEE

SEQ ID NO: 7 - protein, 133 aa, Artificial sequence
  Exemplary recombinant PLK1 substrate peptide.
XEAFDPPLHSTAIXADEEGGPGGXEAFDPPLHSTAIXADEEGGPGGXEAFDPPLHSTAIXADEEGGPGGXEAFDPPLHSTAIXADEEGGPGGXEAFDPPLHSTAIXADEEGGPGGXEAFDPPLHSTAIXADEE

SEQ ID NO: 8 - protein, 138 aa, Artificial sequence
  Exemplary recombinant PLK1 substrate peptide.
GGPGGFEAFDPPLHSTAIFADEEGGPGGFEAFDPPLHSTAIFADEEGGPGGFEAFDPPLHSTAIFADEEGGPGGFEAFDPPLHSTAIFADEEGGPGGFEAFDPPLHSTAIFADEEGGPGGFEAFDPPLHSTAIFADEE

SEQ ID NO: 9 - protein, 179 aa, Artificial sequence
  Exemplary recombinant PLK1 substrate peptide.
XEAFDPPLHSTAIXADEEGGPGGXEAFDPPLHSTAIXADEEGGPGGXEAFDPPLHSTAIXADEEGGPGGXEAFDPPLHSTAIXADEEGGPGGXEAFDPPLHSTAIXADEEGGPGGXEAFDPPLHSTAIXADEEGGPGGXEAFDPPLHSTAIXADEEGGPGGXEAFDPPLHSTAIXADEE

SEQ ID NO: 10 - protein, 418 aa, Homo sapiens
MAPRGRRRPRPHRSEGARRSKNTLERTHSMKDKAGQKCKPIDVFDFPDNSDVSSIGRLGENEKDEETYETFDPPLHSTAIYADEEEFSKHCGLSLSSTPPGKEAKRSSDTSGNEASEIESVKISAKKPGRKLRPISDDSESIEESDTRRKVKSAEKISTQRHEVIRTTASSELSEKPAESVTSKKTGPLSAQPSVEKENLAIESQSKTQKKGKISHDKRKKSRSKAIGSDTSDIVHIWCPEGMKTSDIKELNIVLPEFEKTHLEHQQRIESKVCKAAIATFYVNVKEQFIKMLKESQMLTNLKRKNAKMISDIEKKRQRMIEVQDELLRLEPQLKQLQTKYDELKERKSSLRNAAYFLSNLKQLYQDYSDVQAQEPNVKETYDSSSLPALLFKARTLLGAESHLRNINHQLEKLLDQG

SEQ ID NO: 11 - protein, 25 aa, Artificial sequence
  Consensus polo-box peptide
KWVDYSXKXGXXYQLXXXXXXVXFN

SEQ ID NO: 12 - protein, 5 aa, Artificial sequence
  Exemplary peptide linker.
GGPGG

(19) United States
(12) Patent Application Publication
(10) Pub. No.: US 2011/0076693 A1
(43) Pub. Date: Mar. 31, 2011

(54) METHOD FOR DETECTION AND QUANTIFICATION OF PLK1 EXPRESSION AND ACTIVITY

(75) Inventors: Kyung S. Lee, Gaithersburg, MD (US); Jung Eun Park, Bethesda, MD (US)

(73) Assignee: The U.S.A. as Represented by the Secretary , Dept.of Health and Human Services (the Gov.), Bethesda, MD (US)

(21) Appl. No.: 12/992,887

(22) PCT Filed: May 15, 2009
(86) PCT No.: PCT/US09/44202

§ 371 Date: Nov. 15, 2010

Related U.S. Application Data
(60) Provisional application No. 61/054,032, filed on May 16, 2008.

(51) Int. Cl. G01N 33/573; C07K 14/00; C07K 7/06; C07K 7/08; C12Q 1/48; G01N 33/53

(52) U.S. Cl. 435/7.4; 530/325; 530/350; 530/328; 530/326; 530/324; 530/327; 536/23.1; 435/15; 435/7.1

(57) ABSTRACT

Isolated peptide substrates of Plk1 and nucleic acids encoding these peptides are disclosed. The peptides include two to ten repeats of the amino acid sequence set forth as X₁X₂AX₃X₄X₅PLHSTX₆X₇X₈X₉X₁₀X₁₁X₁₂ (SEQ ID NO: 1), in which within each repeat X₁, X₂, X₆, X₇, X₈, X₉, X₁₀, X₁₁, and X₁₂ are each independently any amino acid or no amino acid, and X₃ and X₄ are each independently any amino acid. Methods of using these peptides to detect Plk1 activity in a sample are also disclosed. In some examples, the method includes contacting a sample with a disclosed peptide substrate of Plk1 in the presence of adenosine triphosphate, or an analog thereof, for a period of time sufficient for Plk1 to phosphorylate the PBIPtide. The presence and/or amount of the phosphorylated and/or the unphosphorylated peptide is detected, thereby detecting and/or quantitating Plk1 kinase activity in the sample.

### CROSS REFERENCE TO RELATED APPLICATION

This application claims the benefit of U.S. Provisional Application No. 61/054,032, filed May 16, 2008, which is incorporated herein by reference in its entirety.

### FIELD OF THE DISCLOSURE

This disclosure relates to Polo Like Kinase 1 (Plk1) substrates and assays for the activity of Plk1 using those substrates.

### BACKGROUND

Cancer is a disease characterized by uncontrolled cell proliferation. This unregulated cellular proliferation can be caused by alterations in the genes controlling the cell cycle. Efforts to develop useful therapies to regulate uncontrolled hyper-proliferative properties of cancer cells have met with limited success.

Members of the polo subfamily of protein kinases have been identified in various eukaryotic organisms, and appear to play pivotal roles in cell proliferation and cell division. A mammalian polo family serine threonine protein kinase, Polo-Like Kinase 1 (Plk1), is expressed at high levels in tumors of various origins (such as breast, ovarian, non-small cell lung, head/neck, colon, endometrial and esophageal carcinomas, and leukemias), and uncontrolled Plk1 expression has been implicated in the development of cancers in humans.

The polo kinase subfamily members are characterized by the presence of a distinct region of homology in the C-terminal non-catalytic domain, termed the polo-box domain (PBD) (Clay et al., Proc. Natl. Acad. Sci. USA. 90:4882-4886, 1993). In mammalian cells, four Plks (from Plk1 to Plk4) exist, but their expression patterns and functions appear to be distinct from each other (Winkles and Albert, Oncogene 24:260-266, 2005). Among these, Plk1 has been a focus of study because of its association with neoplastic transformation of human cells. However, a need remains for assays for specific PLK1 activity.

### SUMMARY OF THE DISCLOSURE

Isolated peptide substrates of Plk1 are disclosed. The disclosed peptide substrates are specific for Plk1, in that that they bind and are phosphorylated by Plk1, but not by the related polo kinases Plk2, Plk3 and Plk4. The disclosed polypeptides are optimized PBIP1 (a natural substrate of Plk1) related peptides with enhanced specificity and sensitivity over the native PBIP1 sequence. The disclosed polypeptides include two to ten consecutive repeats of the amino acid sequence set forth as X₁X₂AX₃X₄X₅PLHSTX₆X₇X₈X₉X₁₀X₁₁X₁₂ (SEQ ID NO: 1), in which within each repeat X₁ is independently any amino acid or no amino acid, X₂ is independently any amino acid or no amino acid, X₃ is independently any amino acid, X₄ is independently any amino acid, X₅ is independently any amino acid, X₆ is independently any amino acid, X₇ is independently any amino acid, X₈ is independently any amino acid or no amino acid, X₉ is independently any amino acid or no amino acid, X₁₀ is independently any amino acid or no amino acid, X₁₁ is independently any amino acid or no amino acid, and X₁₂ is independently any amino acid or no amino acid. The two to ten consecutive repeats of SEQ ID NO: 1 are joined together by peptide linkers that are between two and ten amino acids in length.

Because the disclosed polypeptides can be phosphorylated by Plk1 at the exclusion of other kinases, the polypeptides are specific for Plk1 and can be used to specifically detect Plk1 and/or the kinase activity of Plk1 in a sample, such as a biological sample. Thus, also disclosed are methods for detecting Plk1 kinase activity in a sample, such as a biological sample. In some examples, the method includes contacting a sample with the peptide substrate of Plk1 in the presence of adenosine triphosphate, or an analog thereof, for a period of time sufficient for the Plk1 to phosphorylate the peptide substrate of Plk1, if Plk1 is present in the sample. The presence and/or amount of the phosphorylated peptide is detected, thereby detecting and/or quantitating Plk1 kinase activity in the sample. Methods are also disclosed for detecting a tumor in a subject. In addition, methods are disclosed for identifying inhibitors of Plk1. Agents identified as inhibitors represent potential therapeutic agents for the treatment of cancer.

The foregoing and other features and advantages of the invention will become more apparent from the following detailed description of a several embodiments which proceeds with reference to the accompanying figures.

### BRIEF DESCRIPTION OF THE FIGURES

FIGS. 1A-1B are digital images of Western blots, a schematic representation of a kinase assay and a set of bar graphs showing that a peptide that contains the T78 motif of PBIP1 (a PBIPtide) can be used to quantitate the kinase activity of Plk1 in vitro. FIG. 1A is a set of digital images of Western blots and a schematic of a kinase assay demonstrating that PBIPtides can be used to precipitate Plk1 in vitro, and that PBIPtides are in vitro substrates for Plk1 phosphorylation. FIG. 1A Left, mitotic HeLa lysates were incubated with either bead-immobilized control glutathione S-transferase (GST) or GST-PBIPtide4 in TBSN buffer containing phosphatase inhibitors. Beads were precipitated and washed and then subjected to in vitro kinase assays in the presence of [γ-³²P]-ATP. Samples were separated by sodium dodecyl sulfate polyacrylamide gel electrophoresis (SDS/PAGE), transferred to polyvinylidene fluoride (PVDF), and then exposed (Autorad). The membrane was immunoblotted with the indicated antibodies and then stained with Coomassie (CBB). Note that the anti-p-T78 signal (α-p-T78 panel) is relatively weak because the incubation was carried out in TBSN at 4° C. FIG. 1A Right is schematic diagram illustrating the experimental procedures described in FIG. 1A Left. The ellipsoids depict bead-associated GST-PBIPtide containing the T78 motif. FIG. 1B is a set of digital images of Western blots demonstrating that a PBIPtide can be used to measure the kinase activity of Plk1 from total cellular lysates. A direct Plk1 kinase assay with total cellular lysates using GST-PBIPtides as substrate. Asynchronously growing HeLa cells (Asyn) or cells expressing shRNA directed against control luciferase (shLuc) or Plk1 (shPlk1) were harvested. Where indicated, cells were treated with nocodazole (Noc) for 16 hours to arrest the cells in prometaphase. Total HeLa lysates (100 μg) were prepared in KC-plus buffer and incubated with the control GST or the indicated GST-PBIPtide in the presence of [γ-³²P]-ATP at 30° C. for 30 minutes. The bead-associated GST-PBIPtides were precipitated and washed with KC-plus buffer before boiling with SDS/PAGE sample buffer. The samples were separated, transferred, and then exposed (Autorad). After immunoblotting, the membrane was stained with Coomassie (CBB). The GST-PBIPtide bands were excised, and incorporated 32P was quantified. The signals in the anti-Plk1 and anti-p-T210 immunoblots indicate the amount of Plk1 coprecipitated (Plk1 co-ppt'ed) with the GST-PBIPtide and the level of the p-T210 epitope among the Plk1 precipitates, respectively. Numbers indicate the levels of the p-T78 epitope (γ-p-T78 panel) or 32P incorporation (Autorad) relative to those in the nocodazole (Noc)-treated, control luciferase RNAi (shLuc) cells.

FIGS. 2A-2C are digital images of Western blots and a set of bar graphs showing that Plk1, but not Plk2 or Plk3, phosphorylates and binds to the T78 motif of PBIPtides. FIG. 2A is a set of digital images of Western blots showing that Flag-Plk1 expressed from HeLa cells, but not Flag-Plk2, or Flag-Plk3, can be precipitated with PBIPtides immobilized on agarose beads. 293T lysates from cells expressing control vector, Flag-Plk1, Flag-Plk2, or Flag-Plk3 were prepared in KC-plus buffer and incubated with the indicated GST-PBIPtides immobilized to the GSH-agarose beads. The GST-PBIPtide precipitates were separated by SDS/PAGE, transferred, and then immunoblotted with anti-Plk1 and anti-p-T78 antibodies. Afterward, the membrane was stained with Coomassie (CBB). The level of Plk3 expression is low because of its cytotoxicity. The low levels of the p-T78 signal detected in the control vector-, Plk2-, and Plk3-expressing cells are likely due to the endogenous Plk1 activity. The multiple tiers of the p-T78 signals are due to GST-PBIPtide degradation. Arrowheads indicate Plk1 coprecipitated with GST-PBIPtides. FIGS. 2B and 2C are digital images of Western blots and a set of bar graphs showing that Plk1-dependent phosphorylation of the T78 motif is sufficient to bind to the phospho-peptide binding cleft of the Polo-box Binding Domain (PBD). Plk1-dependent phosphorylation onto the T78 motif of GST-PBIPtide-A6 is sufficient for the PBD binding. FIG. 2B top and middle, Flag-Plk1, Flag-Plk2, and Flag-Plk3 immunoprecipitates prepared from transfected 293T cells were subjected to in vitro kinase assays using both GST-PBIPtide-A6 and casein as substrates in the same reaction. Samples were separated by SDS/PAGE for autoradiography (Autorad). Arrowheads indicate autophosphorylated signals corresponding to Plk1, 2, or 3 in the gels stained with Coomassie (CBB), whereas asterisks denote nonspecific signals. Incorporated 32P into GST-PBIPtide-A6 and casein were quantified (Bottom). FIG. 2C, The Plk1-phosphorylated, bead-bound, GST-PBIPtide-A6 was digested with thrombin, and the resulting soluble 32P-PBIPtide-A6 was incubated with either bead-bound GST-PBD or GST-PBD (H538A K540M) in TBSN buffer. Precipitates were washed and then analyzed as in FIG. 2B.

FIGS. 3A-3E are a schematic representation of an exemplary enzyme-linked immunosorbent assay (ELISA) assay to measure the kinase activity of Plk1 and graphs showing the results of Plk1 kinase assays using various optimized Plk1 substrate peptides. FIG. 3A is a schematic representation of an exemplary Plk1 ELISA assay. The ELISA wells were coated with soluble GST-PBIPtide containing the T78 motif and then reacted with total cellular lysates. The Plk1 activity in the total lysates generates the p-T78 epitope to which Plk1 itself binds. After reaction, Plk1 activity is quantified by incubating the ELISA wells with either anti-p-T78 antibody (to detect the p-T78 epitope generated) or anti-Plk1 antibody (to detect Plk1 bound to the p-T78 epitope), followed by HRP-conjugated secondary antibody (the grey antibody with a black dot). The asterisks indicate 3.3′, 5.5′-tetramethylbenzidine (TMB) substrate and its reaction product, generated by HRP. FIG. 3B is a set of bar graphs demonstrating that PBIPtides containing multiple T78 motifs can be used to measure the kinase activity of Plk1. HeLa cells were silenced for control luciferase (shLuc) or Plk1 (shPlk1) and then treated with nocodazole for 16 hours to arrest the cells in prometaphase (a condition that maximizes Plk1 activity). The lysates were then applied onto the ELISA wells coated with GST-PBIPtide4 or GST-PBIPtide-A6. Buffer indicates no-lysate control. The same lysates were subjected to immunoblotting analysis to determine the level of Plk1 in the lysates. FIG. 3C is a set of graphs demonstrating that Plk1 phosphorylates the T78 motif of the PBIPtide substrate and binds to the phospho-T78 motif in a concentration-dependent manner. Plk1 generates and binds to the p-T78 epitope in a concentration-dependent manner. GST-PBIPtide-A6-coated ELISA wells were incubated with the indicated amount of recombinant Plk1 purified from Sf9 cells. The level of the p-T78 epitope generated and the amount of Plk1 bound to the p-T78 GST-PBIPtide-A6 were quantified by using anti-p-T78 and anti-Plk1 antibodies, respectively. FIG. 3D is a set of bar graphs demonstrating that the PBIPtide, PBIPtide-A₆, can be used to measure the ability of a Plk1-specific inhibitor, such as BI 2536 (BI) to inhibit the kinase activity of Plk1 in total cellular extracts. HeLa cells arrested with nocodazole for 16 hours were additionally treated with either control dimethyl sulfoxide (DMSO) or a Plk1 inhibitor, BI 2536, for 30 minutes before harvest. Total lysates were prepared from these cells and applied to the GST-PBIPtide-A6-coated wells. Buffer indicates no-lysate control. FIG. 3E is a set of bar graphs demonstrating that depletion of Plk1, but not Plk3, drastically diminishes the level of the p-T78 epitope on PBIPtide and PBIPtide-A₆. HeLa cells silenced for control luciferase (shLuc), Plk1 (shPlk1), or Plk3 (shPlk3) were treated with either thymidine (Thy) or nocodazole (Noc) or left untreated for 16 h before harvest. Total cellular lysates prepared from these cells were subjected to ELISAs. Buffer indicates no-lysate control. Because detection of endogenous Plk3 with currently available antibodies was not reliable, efficiency of Plk3 depletion by shPlk3 was determined by using cells transfected with Flag-Plk3.

FIG. 4 is a set of digital images of Western blots and a set of bar graphs showing a tight correlation between Plk1 kinase activity in various mouse tissues as measured by a conventional immunocomplex kinase assay and the disclosed kinase assay.

FIGS. 5A-5C are a set of digital images of xenografted mouse tumors, bar graphs and digital images of Western blots showing direct measurement of in vivo Plk1 kinase activity. FIG. 5A is a set of digital images of xenografted mouse tissue showing tumor formation in athymic mice engrafted with B16 mouse tumor cells (over expressing Plk1). FIG. 5B is a set of bar graphs showing the Plk1 kinase activity of total cellular protein extracted from tumors obtained from the mice shown in FIG. 5A, as measured with the disclosed kinase assay. FIG. 5C is a set of digital images of Western blots showing expression of Plk1 in the tumors obtained from the mice shown in FIG. 5A.

FIG. 6 is a set of digital images of Western blots and bar graphs showing that Plk1 is upregulated in human tumors but not the tissue surrounding the tumors, as measured with the kinase assay disclosed herein.

FIGS. 7A-7C are a set of digital images of Western blots showing that wild-type Plk1, but not a kinase-inactive form, efficiently phosphorylates PBIPtide. FIG. 7A is a set of digital images of Western blots showing that endogenous Plk1 (wild-type) phosphorylates PBIPtides (GST-PBIPtide-Z₄ and GST-PBIPtide₄) and the in vitro Plk1 phospho-transfer target casein. FIG. 7B is a set of digital images of Western blots showing that kinase-inactive Plk1 (K82M) does not phosphorylate Plk1 substrates. Plk1 was immunoprecipitated with anti-GFP antibody from HeLa cells expressing either EGFP-Plk1 or the corresponding kinase-inactive Plk1 (K82M) Immunoprecipitates were then subjected to kinase reactions using GST-PBIPtides as substrates. Samples were separated by SDS/PAGE, exposed (Autorad), and then blotted with anti-p-T78 antibody to examine the level of the p-T78 epitope generated. Later, the same membrane was stained with Coomassie (CBB). Dots indicate the positions of each substrate. FIG. 7C is a set of digital images of Western Blots showing that GST-PBIPtide₄ can precipitate green fluorescent protein (GFP) Plk1 fusion protein. HeLa cells were infected with lentivirus expressing either control shLuc or shPlk1, treated with nocodazole for 16 h where indicated, and then harvested for immunoblotting analyses with the indicated antibodies. The same membrane was stained with Coomassie (CBB). The levels of actin and the CBB staining serve as loading controls.

FIGS. 8A-8B are a set of digital images of Western blots showing that PBIPtides can efficiently precipitate Plk1 and its Xenopus laevis homolog, Plx1, from total cell lysates. FIG. 8A is a set of digital images of Western blots showing that PBIPtides can be used to efficiently immunoprecipitate Plk1. Mitotic HeLa lysates were prepared in KC-plus buffer and incubated with bead-bound GST or GST-PBIPtides. Anti-Plk1 immunoprecipitation with a commercially available anti-Plk1 antibody (N-19; Santa Cruz Biotechnology) was carried out as a comparison. Precipitates were separated and then immunoblotted with the indicated antibodies. Afterward, the same membrane was stained with Coomassie (CBB). FIG. 8B is a set of digital images of Western blots showing that PBIPtides can be used to efficiently immunoprecipitate Plx1 from Xenopus laevis cellular extracts. CSF-arrested egg extracts from Xenopus laevis were diluted in KC-plus buffer and incubated with the indicated ligands immobilized to the beads. Precipitates were washed and then subjected to in vitro kinase reaction in the presence of [γ-³²P]-ATP. The resulting samples were separated by SDS/PAGE, transferred, and then exposed (Autorad). Subsequently, the same membrane was immunoblotted with the indicated antibodies and stained with Coomassie (CBB). Arrows indicate weakly detectable Plx1 precipitated by GST-PBIPtides.

FIG. 9 is a set of bar graphs demonstrating the results of an exemplary Plk1 ELISA using GST-PBIPtides as Plk1 substrate. ELISA wells were coated with the indicated amount of either GST-PBIPtide4 or GST-PBIPtide-A6. The wells were incubated with the designated amount of total cellular lysates prepared from HeLa cells treated with thymidine (S phase) or nocodazole (M phase) for 16 hours. Because of the high sensitivity of the p-T78-based assay as shown in FIG. 3B, only the p-T78 antibody was used for analyses. With a given amount of total cellular lysates, the reactions in were saturated with 0.3 μg of GST-PBIPtide-A6. Under the conditions used, all of the reactions were terminated in 10 seconds because the signals for the 20-μg lysates were already saturated.

FIG. 10 is a digital image of Western blots showing Anti-Plk1 immunocomplex kinase assays with various mouse tissues using casein as substrate. Results demonstrate a tight correlation between the disclosed Plk1 assay and the conventional anti-Plk1 immunocomplex kinase assay. Anti-Plk1 immunoprecipitates from various tissues were subjected to in vitro kinase assays under the same conditions as in FIG. 4A except that GST-PBIPtide-A6 was used as substrate. Asterisk indicates that only half the amount of total lysates (1 mg) and anti-Plk1 antibody (3 μg) was used for ovary immunoprecipitation because of the limited amount of the tissue. Note that the relative levels of GST-PBIPtide-A6 phosphorylation by immunoprecipitated Plk1 are in line with those of the casein phosphorylation in FIG. 4A.

FIG. 11A-11C are a set of digital images and a bar graph showing the direct measurement of in vivo Plk1 kinase activity in xenografted mouse tumors using Plk1 ELISA assay. FIG. 11A is a digital image of athymic nude mice that were subcutaneously grafted with 4×10⁶ cells of B16 mouse tumor line. At the indicated days, mice were sacrificed and the resulting tumors were surgically removed for subsequent analyses. Bar, 1 cm. FIG. 11B is a digital image of tumor sections from the tumors in FIG. 11A were prepared and subjected to hematoxylin and eosin stain (H&E) and bromodeoxyuridine (BrdU) stainings. Largely correlating with the levels of Plk1 activity in FIG. 11C, the BrdU-positive, proliferating, cells are highly concentrated at the growing edge of the tumors during the early stages of tumorigenesis (2, 4, and 8 days). In contrast, the 12 and 16-day tumors exhibit sparsely populated proliferating cells, suggesting a diminished level of cell proliferation activity. FIG. 11C, top, total proteins prepared from the tumors in FIG. 11A were separated by SDS-PAGE for anti-Plk1 immunoblotting analyses and then stained with Coomassie (CBB) for loading controls. FIG. 11C, bottom, Plk1 ELISA assays were carried out with 20 μg of the same total lysates using GST-fused PBIPtide-A6 form as a Plk1 substrate. Bars, standard deviation. Note that the levels of the BrdU-positive cells in FIG. 11B correlate with those of Plk1 expression and activity in FIG. 11C.

FIG. 12A-12B is a set of digital images of mouse tissue and Western blots and a bar graph showing a close correlation between the levels of Plk1 expression and activity and those of mitotic Cyclin B1 in a B16-derived tumor. FIG. 12A, athymic mice were subcutaneously grafted with B16 tumor cells. One of the large tumors was surgically removed and then divided into 9 sections. Bar, 1 cm. FIG. 12B, top, total proteins prepared from each section were subjected to immunoblotting analyses with the indicated antibodies and then stained with Coomassie (CBB). Asterisk, a cross-reacting protein with anti-Cyclin B1 antibody. FIG. 12B, bottom Plk1 ELISA assays were carried out with 20 μg of the same total lysates using GST-PBIPtide-A6 form as a Plk1 substrate. Likely due to differences in the degree of senescence among different parts of the tumors, the levels of Plk1 activities vary significantly in #1 to #9 samples. However, it should be noted that the levels of Plk1 expression and activity tightly correlate with those of cyclin B1, suggesting that Plk1 is a reliable marker for cell proliferation. Bars, standard deviation.

FIG. 13A-13B is a set of bar graphs showing the quantification of Plk1 activity in tumor and normal tissues from various head and neck cancer patients. T, tumor tissues; N, normal tissues. Bars, standard deviation. T1-T4, size and/or extent of the primary tumor; N0, no regional lymph node involvement; N1-N3, extent of spread into regional lymph nodes; M0, no distant metastasis; Mx, distant metastasis can not be evaluated.

FIG. 14A-14C is a set of bar graphs showing the quantification of Plk1 activity in tumor and the corresponding normal tissues in three major cancer types among South Korean population.

### SEQUENCES

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand.

SEQ ID NOs: 1-9 show the amino acid sequence of exemplary peptide substrates for Plk1 kinase.

SEQ ID NO: 10 is the amino acid sequence of an exemplary form of PBIP1.

SEQ ID NO: 11 shows the consensus amino acid sequence of a polo-box.

SEQ ID NO: 12 shows an exemplary amino acid sequence a peptide linker.

### DETAILED DESCRIPTION

### I. Terms

Unless otherwise noted, technical terms are used according to conventional usage. Definitions of common terms in molecular biology can be found in Benjamin Lewin, Genes VII, published by Oxford University Press, 1999; Kendrew et al. (eds.), The Encyclopedia of Molecular Biology, published by Blackwell Science Ltd., 1994; and Robert A. Meyers (ed.), Molecular Biology and Biotechnology: a Comprehensive Desk Reference, published by VCH Publishers, Inc., 1995; and other similar references.

As used herein, the singular forms “a,” “an,” and “the,” refer to both the singular as well as plural, unless the context clearly indicates otherwise. For example, the term “a peptide” includes single or plural peptides and can be considered equivalent to the phrase “at least one peptide.”

As used herein, the term “comprises” means “includes.” Thus, “comprising a peptide” means “including a peptide” without excluding other elements.

It is further to be understood that all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described below. In case of conflict, the present specification, including explanations of terms, will control. In addition, the materials, methods, and examples are illustrative only and not intended to be limiting.

To facilitate review of the various embodiments of the invention, the following explanations of terms are provided:

Animal: Living multi-cellular vertebrate organisms, a category that includes, for example, mammals and birds. The term mammal includes both human and non-human mammals. Similarly, the term “subject” includes both human and veterinary subjects.

Antibody: A polypeptide ligand which include a light chain and/or heavy chain immunoglobulin variable region which specifically binds an epitope of an antigen, such as the Plk1 substrate peptides disclosed herein. In other examples, an antibody is an antibody that specifically binds an epitope of a Plk1 peptide. The term “specifically binds” refers to, with respect to an antigen such as a Plk1 substrate peptide (for example a phosphorylated Plk1 substrate peptide), the preferential association of an antibody or other ligand, in whole or part, with the Plk1 substrate peptide. A specific binding agent binds substantially only to a defined target, such as the Plk1 substrate peptide, for example a phosphorylated Plk1 substrate peptide and not to a non-phosphorylated Plk1 substrate peptide. Thus, in one example a Plk1 substrate peptide specific antibody is an antibody that specifically binds to a Plk1 substrate peptide when the peptide is not phosphorylated and not the same Plk1 substrate peptide when the Plk1 substrate peptide is phosphorylated. Conversely, a phosphorylated Plk1 substrate peptide specific antibody is an antibody that specifically binds to a phosphorylated Plk1 substrate peptide (for example phosphorylated on one or more threonine residues) and not the same Plk1 substrate peptide when the Plk1 substrate peptide is not phosphorylated. In another example, a Plk1 specific antibody is an antibody that specifically binds to Plk1. It is recognized that a minor degree of non-specific interaction may occur between a molecule, such as a antibody, and a non-target polypeptide. Nevertheless, specific binding can be distinguished as mediated through specific recognition of the antigen. Although selectively reactive antibodies bind antigen, they can do so with low affinity. Specific binding typically results in greater than 2-fold, such as greater than 5-fold, greater than 10-fold, or greater than 100-fold increase in amount of bound antibody or other ligand (per unit time) to a target polypeptide as compared to a non-target polypeptide. A variety of immunoassay formats are appropriate for selecting antibodies specifically immunoreactive with a particular protein. For example, solid-phase ELISA immunoassays are routinely used to select monoclonal antibodies specifically immunoreactive with a protein. See Harlow & Lane, Antibodies, A Laboratory Manual, Cold Spring Harbor Publications, New York (1988), for a description of immunoassay formats and conditions that can be used to determine specific immunoreactivity.

Antibodies can be composed of a heavy and a light chain, each of which has a variable region, termed the variable heavy (VH) region and the variable light (VL) region. Together, the VH region and the VL region are responsible for binding the antigen recognized by the antibody. This includes intact immunoglobulins and the variants and portions of them well known in the art, such as Fab′ fragments, F(ab)′2 fragments, single chain Fv proteins (“scFv”), and disulfide stabilized Fv proteins (“dsFv”). A scFv protein is a fusion protein in which a light chain variable region of an immunoglobulin and a heavy chain variable region of an immunoglobulin are bound by a linker, while in dsFvs, the chains have been mutated to introduce a disulfide bond to stabilize the association of the chains. The term also includes recombinant forms such as chimeric antibodies (for example, humanized murine antibodies), heteroconjugate antibodies (such as, bispecific antibodies). See also, Pierce Catalog and Handbook, 1994-1995 (Pierce Chemical Co., Rockford, Ill.); Kuby, Immunology, 3rd Ed., W.H. Freeman & Co., New York, 1997.

A “monoclonal antibody” is an antibody produced by a single clone of B-lymphocytes or by a cell into which the light and heavy chain genes of a single antibody have been transfected. Monoclonal antibodies are produced by methods known to those of skill in the art, for instance by making hybrid antibody-forming cells from a fusion of myeloma cells with immune spleen cells. These fused cells and their progeny are termed “hybridomas.” Monoclonal antibodies include humanized monoclonal antibodies.

Cancer: A malignant disease characterized by the abnormal growth and differentiation of cells. “Metastatic disease” refers to cancer cells that have left the original tumor site and migrate to other parts of the body for example via the bloodstream or lymph system.

Examples of hematological tumors include leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia, and myelodysplasia.

Examples of solid tumors, such as sarcomas and carcinomas, include fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, colon carcinoma, lymphoid malignancy, pancreatic cancer, breast cancer (such as adenocarcinoma), lung cancers, gynecological cancers (such as, cancers of the uterus (e.g., endometrial carcinoma), cervix (e.g., cervical carcinoma, pre-tumor cervical dysplasia), ovaries (e.g., ovarian carcinoma, serous cystadenocarcinoma, mucinous cystadenocarcinoma, endometrioid tumors, celioblastoma, clear cell carcinoma, unclassified carcinoma, granulosa-thecal cell tumors, Sertoli-Leydig cell tumors, dysgerminoma, malignant teratoma), vulva (e.g., squamous cell carcinoma, intraepithelial carcinoma, adenocarcinoma, fibrosarcoma, melanoma), vagina (e.g., clear cell carcinoma, squamous cell carcinoma, botryoid sarcoma), embryonal rhabdomyosarcoma, and fallopian tubes (e.g., carcinoma)), prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, and CNS tumors (such as a glioma, astrocytoma, medulloblastoma, craniopharyngioma, ependymoma, pinealoma, hemangioblastoma, acoustic neuroma, oligodendroglioma, menangioma, melanoma, neuroblastoma and retinoblastoma), and skin cancer (such as melanoma and non-melanoma). In particular examples, a cancer associated with Plk1 expression is breast cancer, ovarian cancer, non-small cell lung cancer, head/neck cancer, colon cancer, endometrial cancer, esophageal carcinoma, or leukemia.

Contacting: Placement in direct physical association, which can include both in solid and liquid form. Contacting can occur in vitro with for example with samples, such as biological samples, for example isolated cells or cell free extracts, such as cell lysates, or in vivo by administering to a subject. In some examples, a sample is contacted with a Plk1 substrate peptide, such as the peptides disclosed herein. In some examples, a sample is contacted with Plk1.

Control: A reference standard. In some examples, a control can be a known value indicative of basal kinase activity of Plk1 for a peptide substrate, such as the peptide substrates disclosed herein. In other examples, a control in the kinase activity of Plk1 in a sample not treated with a test agent. A difference between a test sample and a control can be an increase or conversely a decrease. The difference can be a qualitative difference or a quantitative difference, for example a statistically significant difference. In some examples, a difference is an increase or decrease, relative to a control, of at least about 10%, such as at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, at least about 500%, or greater then 500%.

Complex (complexed): Two proteins, or fragments or derivatives thereof, one protein (or fragment or derivative) and a non-protein compound, are said to form a complex when they measurably associate with each other in a specific manner. In some examples, a complex is the complex formed between a kinase, such as Plk1 and peptide substrate of the kinase, such as the peptides disclosed herein. In another example, a complex is the complex formed between Plk1 and an antibody that specifically binds to Plk1. In another example, a complex is the complex formed between an antibody that specifically binds to a Plk1 substrate peptide and the substrate peptide (such as the peptides disclosed herein).

Heterologous: With reference to a molecule, such as a Plk1 substrate peptide or a linker, “heterologous” refers to molecules that are not normally associated with each other, for example as a single molecule. Thus, a “heterologous” peptide linker is a peptide linker attached to another molecule to which the peptide linker is usually not found in association with in nature, such as in a wild-type molecule. For example, two repeated amino acid sequences of a Plk1 substrate peptide, for example the amino acid sequence according to SEQ ID NO: 1 can be attached to a heterologous linker (for example linked by the peptide linker) that they are not naturally attached to, for example to join the repeating sequences of the Plk1 substrate peptide.

Host cells: Cells in which a vector can be propagated and its DNA expressed, for example DNA encoding the Plk1 substrate peptides disclosed herein. The cell may be prokaryotic or eukaryotic. The term also includes any progeny of the subject host cell. It is understood that all progeny may not be identical to the parental cell since there may be mutations that occur during replication. However, such progeny are included when the term “host cell” is used.

Inhibitor of Plk1 kinase: A compound that, when applied to a cell or cell free system, exhibits a measurable inhibitory activity against the function of Plk1. In particular, such effects include any or all of the following: a modification in the subcellular localization of Plk1; a modification in binding affinity of the polo-box domain for one or more specific binding partners; a change in the phosphorylating activity of the Plk1 kinase domain; or an alteration (either stimulation or inhibition) in the stability of Plk1. In some examples, a compound with Plk1 kinase inhibitory activity is identified using the assays disclosed herein.

Detect: To determine if an agent (such as a signal or particular molecule) is present or absent. In some examples, this can further include quantification. In some examples, the disclosed assays are used to detect the kinase activity of Plk1.

Detectable Label: An agent capable of detection, for example by spectrophotometry, flow cytometry, or microscopy. For example, a label can be attached to a specific binding agent, such as an antibody or a protein, thereby permitting detection of a biomolecule bound to the specific binding agent, for example the peptides disclosed herein. Specific, non-limiting examples of labels include fluorescent tags, enzymatic linkages, and radioactive isotopes and nanoparticles, such as semiconductor nanocrystals. Methods for labeling are discussed for example in Sambrook et al. (Molecular Cloning: A Laboratory Manual, Cold Spring Harbor, N.Y., 1989) and Ausubel et al. (In Current Protocols in Molecular Biology, John Wiley & Sons, New York, 1998).

Electromagnetic radiation: A series of electromagnetic waves that are propagated by simultaneous periodic variations of electric and magnetic field intensity, and that includes radio waves, infrared, visible light, ultraviolet light, X-rays and gamma rays. In particular examples, electromagnetic radiation is emitted by a laser, which can possess properties of monochromaticity, directionality, coherence, polarization, and intensity. Lasers are capable of emitting light at a particular wavelength (or across a relatively narrow range of wavelengths), for example such that energy from the laser can excite one fluorophore with a specific excitation wavelength (for example a fluorophore attached to a Plk1 substrate peptide) but not excite a second fluorophore (for example a fluorophore attached to a Plk1 substrate peptide specific binding agent, such as an antibody or isolated Plk1) with a specific excitation wavelength different and distinct from the excitation wavelength on the first fluorophore.

Emission or emission signal: The light of a particular wavelength generated from a source, for example a fluorophore attached to a peptide protein, such as the Plk1 substrate peptides disclosed herein. In particular examples, an emission signal is emitted from a fluorophore, after the fluorophore absorbs light at its excitation wavelength(s).

Excitation or excitation signal: The light of a particular wavelength necessary and/or sufficient to excite an electron transition to a higher energy level. In particular examples, an excitation is the light of a particular wavelength necessary and/or sufficient to excite a fluorophore (such as a fluorophore attached to a Plk1 substrate peptide disclosed herein), to a state such that the fluorophore will emit a different (such as a longer) wavelength of light then the wavelength of light from the excitation signal.

Expression Control Sequences: Nucleic acid sequences that regulate the expression of a heterologous nucleic acid sequence to which it is operatively linked. Expression control sequences are operatively linked to a nucleic acid sequence when the expression control sequences control and regulate the transcription and, as appropriate, translation of the nucleic acid sequence. Thus expression control sequences can include appropriate promoters, enhancers, transcription terminators, a start codon (ATG) in front of a protein-encoding gene, splicing signal for introns, maintenance of the correct reading frame of that gene to permit proper translation of mRNA, and stop codons. The term “control sequences” is intended to include, at a minimum, components whose presence can influence expression, and can also include additional components whose presence is advantageous, for example, leader sequences and fusion partner sequences. Expression control sequences can include a promoter.

A promoter is a minimal sequence sufficient to direct transcription. Also included are those promoter elements which are sufficient to render promoter-dependent gene expression controllable for cell-type specific, tissue-specific, or inducible by external signals or agents; such elements may be located in the 5′ or 3′ regions of the gene. Both constitutive and inducible promoters are included (see for example, Bitter et al., Methods in Enzymology 153:516-544, 1987). For example, when cloning in bacterial systems, inducible promoters such as pL of bacteriophage lambda, plac, ptrp, ptac (ptrp-lac hybrid promoter) and the like may be used. In one embodiment, when cloning in mammalian cell systems, promoters derived from the genome of mammalian cells (such as metallothionein promoter) or from mammalian viruses (such as the retrovirus long terminal repeat; the adenovirus late promoter; the vaccinia virus 7.5K promoter) can be used. Promoters produced by recombinant DNA or synthetic techniques may also be used to provide for transcription of the nucleic acid sequences.

A polynucleotide can be inserted into an expression vector that contains a promoter sequence which facilitates the efficient transcription of the inserted genetic sequence of the host. The expression vector typically contains an origin of replication, a promoter, as well as specific nucleic acid sequences that allow phenotypic selection of the transformed cells.

For a period of time sufficient: A phrase used to describe a period of time in which a desired activity occurs, for example the time it takes for a kinase such as Plk1, to phosphorylate a peptide substrate, such as the Plk1 substrate peptides disclosed herein. It is appreciated that the time period can be varied based on the concentration of the reagents used and other factors.

Fluorophore: A chemical compound, which when excited by exposure to a particular stimulus, such as a defined wavelength of light, emits light (fluoresces), for example at a different wavelength (such as a longer wavelength of light).

Fluorophores are part of the larger class of luminescent compounds. Luminescent compounds include chemiluminescent molecules, which do not require a particular wavelength of light to luminesce, but rather use a chemical source of energy. Therefore, the use of chemiluminescent molecules (such as aequorin) can eliminate the need for an external source of electromagnetic radiation, such as a laser.

Examples of particular fluorophores that can be used in the methods and for attachment to the Plk1 peptides disclosed herein are provided in U.S. Pat. No. 5,866,366 to Nazarenko et al., such as 4-acetamido-4′-isothiocyanatostilbene-2,2′ disulfonic acid, acridine and derivatives such as acridine and acridine isothiocyanate, 5-(2′-aminoethyl)aminonaphthalene-1-sulfonic acid (EDANS), 4-amino-N-[3-vinylsulfonyl)phenyl]naphthalimide-3,5 disulfonate (Lucifer Yellow VS), N-(4-anilino-1-naphthyl)maleimide, anthranilamide, Brilliant Yellow, coumarin and derivatives such as coumarin, 7-amino-4-methylcoumarin (AMC, Coumarin 120), 7-amino-4-trifluoromethylcouluarin (Coumaran 151); cyanosine; 4′,6-diaminidino-2-phenylindole (DAPI); 5′,5″-dibromopyrogallol-sulfonephthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5[dimethylamino]naphthalene-1-sulfonyl chloride (DNS, dansyl chloride); 4-dimethylaminophenylazophenyl-4′-isothiocyanate (DABITC); eosin and derivatives such as eosin and eosin isothiocyanate; erythrosin and derivatives such as erythrosin B and erythrosin isothiocyanate; ethidium; fluorescein and derivatives such as 5-carboxyfluorescein (FAM), 5-(4,6-dichlorotriazin-2-yl)aminofluorescein (DTAF), 2′7′-dimethoxy-4′5′-dichloro-6-carboxyfluorescein (JOE), fluorescein, fluorescein isothiocyanate (FITC), and QFITC(XRITC); fluorescamine; IR144; IR1446; Malachite Green isothiocyanate; 4-methylumbelliferone; ortho cresolphthalein; nitrotyrosine; pararosaniline; Phenol Red; B-phycoerythrin; o-phthaldialdehyde; pyrene and derivatives such as pyrene, pyrene butyrate and succinimidyl 1-pyrene butyrate; Reactive Red 4 (Cibacron™ Brilliant Red 3B-A); rhodamine and derivatives such as 6-carboxy-X-rhodamine (ROX), 6-carboxyrhodamine (R6G), lissamine rhodamine B sulfonyl chloride, rhodamine (Rhod), rhodamine B, rhodamine 123, rhodamine X isothiocyanate, sulforhodamine B, sulforhodamine 101 and sulfonyl chloride derivative of sulforhodamine 101 (Texas Red); N,N,N′,N′-tetramethyl-6-carboxyrhodamine (TAMRA); tetramethyl rhodamine; tetramethyl rhodamine isothiocyanate (TRITC); riboflavin; rosolic acid and terbium chelate derivatives; LightCycler Red 640; Cy5.5; and Cy56-carboxyfluorescein; 5-carboxyfluorescein (5-FAM); boron dipyrromethene difluoride (BODIPY); N,N,N′,N′-tetramethyl-6-carboxyrhodamine (TAMRA); acridine, stilbene, -6-carboxy-fluorescein (HEX), TET (Tetramethyl fluorescein), 6-carboxy-X-rhodamine (ROX), Texas Red, 2′,7′-dimethoxy-4′,5′-dichloro-6-carboxyfluorescein (JOE), Cy3, Cy5, VIC® (Applied Biosystems), LC Red 640, LC Red 705, Yakima yellow amongst others.

Other suitable fluorophores include those known to those skilled in the art, for example those available from Molecular Probes (Eugene, Oreg.). In particular examples, a fluorophore is used as a donor fluorophore or as an acceptor fluorophore.

“Acceptor fluorophores” are fluorophores which absorb energy from a donor fluorophore, for example in the range of about 400 to 900 nm (such as in the range of about 500 to 800 nm). Acceptor fluorophores generally absorb light at a wavelength which is usually at least 10 nm higher (such as at least 20 nm higher), than the maximum absorbance wavelength of the donor fluorophore, and have a fluorescence emission maximum at a wavelength ranging from about 400 to 900 nm. Acceptor fluorophores have an excitation spectrum overlapping with the emission of the donor fluorophore, such that energy emitted by the donor can excite the acceptor. Ideally, an acceptor and a donor fluorophore are capable of being attached to a peptide, such as the peptides disclosed herein, Plk1 and/or an antibody.

In some examples, a fluorophore is detectable label, such as a detectable label attached to an isolated Plk1, an antibody, or a Plk1 substrate peptide disclosed herein.

High throughput technique: Through this process, one can rapidly identify active compounds, antibodies or genes which affect a particular biomolecular pathway, for example pathways in which Plk1 is involved. In certain examples, combining modern robotics, data processing and control software, liquid handling devices, and sensitive detectors, high throughput techniques allows the rapid screening of potential pharmaceutical agents in a short period of time, for example using the assays disclosed herein.

Inhibitor (for example, of kinase activity, such as Plk1 kinase activity): A substance capable of inhibiting to some measurable extent, for example the kinase activity of a protein, such as Plk1 kinase activity. In disclosed examples, inhibition of Plk1 kinase activity is measured in the assays disclosed herein.

Isolated: An “isolated” biological component, such as a peptide (for example a Plk1 substrate peptide), cell (for example a host cell that includes a nucleic acid encoding a Plk1 substrate peptide), nucleic acid (for example a nucleic acid encoding a Plk1 substrate peptide) has been substantially separated, produced apart from, or purified away from other biological components in the cell of the organism in which the component naturally occurs, for instance, other chromosomal and extrachromosomal DNA and RNA, and proteins. Nucleic acids, peptides and proteins that have been “isolated” thus include nucleic acids and proteins purified by standard purification methods. The term also embraces nucleic acids, peptides and proteins prepared by recombinant expression in a cell as well as chemically synthesized peptide and nucleic acids. The term “isolated” or “purified” does not require absolute purity; rather, it is intended as a relative term. Thus, for example, an isolated peptide preparation is one in which the peptide or protein is more enriched than the peptide or protein is in its natural environment within a cell. Preferably, a preparation is purified such that the protein or peptide represents at least 50% of the total peptide or protein content of the preparation, such as at least 50%, at least 60%, at least 70%, at least 80%, at least 90%, at least 95%, or even at least 99% of the peptide or protein concentration.

Kinase: An enzyme that transfers a phosphate group, usually from ATP to a substrate, such as a peptide. Different families of kinases are capable of transferring a phosphate to different residue types. For example, serine/threonine kinases such as Plk1 kinase can transfer a phosphate from ATP to a serine or threonine residue of a suitable substrate, such as the Plk1 substrate peptides disclosed herein. Kinase inactive refers to a kinase that has been inactivated, for example by modification, such as genetic modification (for example be mutation of one of the catalytic residues) or chemical modification.

Kinase (phosphorylating) activity: Measurable phosphorylating activity of a protein (a kinase). “Phosphorylation” is the addition of a phosphate to a protein or peptide, typically by a kinase. In some examples, kinase activity is the kinase activity of Plk1. The kinase activity of Plk1 is the ability of Plk1 to transfer a phosphate from a nucleotide triphosphate, such as ATP, to a substrate, such as the Plk1 substrate peptides disclosed herein. The kinase activity of Plk1 can be detected and/or quantified using the assays disclosed herein.

Linker: A compound or moiety that acts as a molecular bridge to operably link two different molecules such as two peptides, repeated sequences of a peptides or even a peptide with another molecule (such as a molecule of a solid support, for example a bead or multiwell plate or a detectable label, such as the labels described herein), wherein one portion of the linker is operably linked to a first molecule, and wherein another portion of the linker is operably linked to a second molecule and generally the linker is heterologous to the first and second melecuels. In some examples, a linker is a polypeptide, such as a polypeptide that is between about two amino acid residues and about ten amino acid residues in length. In the case of peptide linker connecting two peptides, the peptide linker can be transcribed from a single piece of nucleic acid that encodes the two peptide and the linker. In some embodiments, there are no particular size or content limitations for the linker so long as it can fulfill its purpose as a molecular bridge. Linkers are known to those skilled in the art to include, but are not limited to, chemical chains, chemical compounds, carbohydrate chains, peptides, haptens, and the like. The linkers can include, but are not limited to, homobifunctional linkers and hetero-bifunctional linkers. Hetero-bifunctional linkers, well known to those skilled in the art, contain one end having a first reactive functionality to specifically link a first molecule, and an opposite end having a second reactive functionality to specifically link to a second molecule. Depending on such factors as the molecules to be linked, and the conditions in which the method of detection is performed, the linker can vary in length and composition for optimizing such properties as flexibility, stability, and resistance to certain chemical and/or temperature parameters. In particular examples, a linker is the combination of streptavidin or avidin and biotin. In other examples, a linker is the combination of GST and glutathione. In a particular example, a peptide linker is the amino acid sequence set forth as GGPGG (SEQ ID NO: 12)

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame. In other examples, a molecule is “operably linked” to another molecule when the two molecules are connected by a linker, for example a linker connecting a peptide to another molecule, such as solid support or a detectable label, or linker connecting two peptides, such as the Plk1 substrate peptides disclosed herein.

Nucleic acid: A polymer composed of nucleotide units (ribonucleotides, deoxyribonucleotides, related naturally occurring structural variants, and synthetic non-naturally occurring analogs thereof) linked via phosphodiester bonds, related naturally occurring structural variants, and synthetic non-naturally occurring analogs thereof. Thus, the term includes nucleotide polymers in which the nucleotides and the linkages between them include non-naturally occurring synthetic analogs, such as, for example and without limitation, phosphorothioates, phosphoramidates, methyl phosphonates, chiral-methyl phosphonates, 2-O-methyl ribonucleotides, peptide-nucleic acids (PNAs), and the like. Such polynucleotides can be synthesized, for example, using an automated DNA synthesizer. The term “oligonucleotide” typically refers to short polynucleotides, generally no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

Conventional notation is used herein to describe nucleotide sequences: the left-hand end of a single-stranded nucleotide sequence is the 5′-end; the left-hand direction of a double-stranded nucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand;” sequences on the DNA strand having the same sequence as an mRNA transcribed from that DNA and which are located 5′ to the 5′-end of the RNA transcript are referred to as “upstream sequences;” sequences on the DNA strand having the same sequence as the RNA and which are 3′ to the 3′ end of the coding RNA transcript are referred to as “downstream sequences.”

“cDNA” refers to a DNA that is complementary or identical to an mRNA, in either single stranded or double stranded form.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA produced by that gene produces the protein in a cell or other biological system. Both the coding strand, the nucleotide sequence of which is identical to the mRNA sequence and is usually provided in sequence listings, and non-coding strand, used as the template for transcription, of a gene or cDNA can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

“Recombinant nucleic acid” refers to a nucleic acid having nucleotide sequences that are not naturally joined together. This includes nucleic acid vectors comprising an amplified or assembled nucleic acid which can be used to transform a suitable host cell. A host cell that comprises the recombinant nucleic acid is referred to as a “recombinant host cell.” The gene is then expressed in the recombinant host cell to produce, for example a “recombinant polypeptide.” A recombinant nucleic acid may serve a non-coding function (for example a promoter, origin of replication, ribosome-binding site, etc.) as well.

A first sequence is an “antisense” with respect to a second sequence if a polynucleotide whose sequence is the first sequence specifically hybridizes with a polynucleotide whose sequence is the second sequence.

Nucleotide: The fundamental unit of nucleic acid molecules. A nucleotide includes a nitrogen-containing base attached to a pentose monosaccharide with one, two, or three phosphate groups attached by ester linkages to the saccharide moiety.

The major nucleotides of DNA are deoxyadenosine 5′-triphosphate (dATP or A), deoxyguanosine 5′-triphosphate (dGTP or G), deoxycytidine 5′-triphosphate (dCTP or C) and deoxythymidine 5′-triphosphate (dTTP or T). The major nucleotides of RNA are adenosine 5′-triphosphate (ATP or A), guanosine 5′-triphosphate (GTP or G), cytidine 5′-triphosphate (CTP or C) and uridine 5′-triphosphate (UTP or U).

Nucleotides include those nucleotides containing modified bases, modified sugar moieties and modified phosphate backbones, for example as described in U.S. Pat. No. 5,866,336 to Nazarenko et al. (herein incorporated by reference).

Examples of modified base moieties which can be used to modify nucleotides at any position on its structure include, but are not limited to: 5-fluorouracil, 5-bromouracil, 5-chlorouracil, 5-iodouracil, hypoxanthine, xanthine, acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N˜6-sopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid, 5-methyl-2-thiouracil, 3-(3-amino-3-N-2-carboxypropyl) uracil, and 2,6-diaminopurine.

Examples of modified sugar moieties which may be used to modify nucleotides at any position on its structure include, but are not limited to: arabinose, 2-fluoroarabinose, xylose, and hexose, or a modified component of the phosphate backbone, such as phosphorothioate, a phosphorodithioate, a phosphoramidothioate, a phosphoramidate, a phosphordiamidate, a methylphosphonate, an alkyl phosphotriester, or a formacetyl or analog thereof.

PBIP1: Polo Box Interacting Protein 1 (PBIP1, also known as KLIP1, MLF1IP, and CENP-50) is a protein that specifically binds to the polo-box domain (PBD) of Plk1 protein. Interactions with Plk1 are modulated by the N-terminus of PBIP1, while the C-terminus modulates dimerization and kinetochore localization. Plk1 phosphorylates PBIP1 on the threonine residue number 78 (T78), which is shown in the sequence below. An exemplary sequence of PBIP1 as available Apr. 4, 2008, at GENBANK® accession no. NP_{—}078905 has the amino acid sequence as set forth as:

MAPRGRRRPRPHRSEGARRSKNTLERTHSMKDKAGQKCKPIDVFDFPDNSD VSSIGRLGENEKDEETYETFDPPLHSTAIYADEEEFSKHCGLSLSSTPPGKEAK RSSDTSGNEASEIESVKISAKKPGRKLRPISDDSESIEESDTRRKVKSAEKISTQ RHEVIRTTASSELSEKPAESVTSKKTGPLSAQPSVEKENLAIESQSKTQKKGKI SHDKRKKSRSKAIGSDTSDIVHIWCPEGMKTSDIKELNIVLPEFEKTHLEHQQ RIESKVCKAAIATFYVNVKEQFIKMLKESQMLTNLKRKNAKMISDIEKKRQR MIEVQDELLRLEPQLKQLQTKYDELKERKSSLRNAAYFLSNLKQLYQDYSD VQAQEPNVKETYDSSSLPALLFKARTLLGAESHLRNINHQLEKLLDQG (SEQ ID NO: 10, T78 is indicated in bold). The T78 motif of PBIP1 includes residues 64-85 of PBIP1.

Peptide, Polypeptide, and/or Protein: Any compound composed of amino acids, amino acid analogs, chemically bound together Amino acids generally are chemically bound together via amide linkages (CONH). Additionally, amino acids may be bound together by other chemical bonds. For example, the amino acids may be bound by amine linkages. Peptides include oligomers of amino acids, amino acid analog, or small and large peptides, including polypeptides or proteins. In some examples, a peptide is a peptide substrate of Plk1, such as the peptide substrates of Plk1 disclosed herein.

Phospho-peptide or phospho-protein: A peptide or protein in which one or more phosphate moieties are covalently linked to amino acid residue, or amino acid analogs. Typically, these are serine, threonine, tyrosine, aspartic acid or histidine. Alternatively, a phospho-peptide may be constructed with non-natural or synthetic amino acids, in which the phosphate is covalently linked to the non-natural or synthetic amino acid. A peptide can be phosphorylated at multiple or single sites. Sometimes it is desirable for the phospho-peptide to be phosphorylated at one site regardless of the presence of multiple potential phosphorylation sites. In vivo the transfer of a phosphate to a peptide is accomplished by a kinase exhibiting kinase activity. In some examples, a peptide is a substrate of Plk1, such as the peptides disclosed herein, that is phosphorylated on a threonine residue by Plk1.

Polo-like kinase: A member of a family of serine/threonine protein kinases that are characterized by the presence of a distinct region of homology in the C-terminal non-catalytic domain of the kinase. This domain is termed the polo-box, and plays a role in the subcellular localization of polo-like kinase proteins. The name of this family of kinases is derived form the polo gene, which encodes the first polo-like kinase identified; polo is a Drosophila gene.

Members of the polo subfamily of protein kinases (e.g., Cdc5, Polo, Plk1ₘₐₘₘₐₗᵢₐₙ, Plo1p, Snk, FNK/Prk, Plx1, Tbplk, and Plk1_{C. elegans}) have been identified in various eukaryotic organisms. These kinases are known to play pivotal roles in cell division and proliferation. Studies in various organisms have shown that polo kinases regulate diverse cellular and biochemical events at multiple stages of M phase. These include centrosome maturation, bipolar spindle formation, and activation of anaphase promoting complex (APC).

Specific examples of sequences of polo-like kinase 1 and its homologues include those disclosed in the following GENBANK® Accession Nos.: P32562 (S. cerevisiae Cdc5); P50528 (S. pombe Plo1); P52304 (D. melanogaster Polo); P34331 (C. elegans Ykz4); P53350 (H. sapiens Plk1); Q07832 (M. musculus Plk1); and CAA02714 (H. sapiens serine-threonine kinase) as available Apr. 4, 2008. These sequences are incorporated herein by reference. An exemplary amino acid sequence of human Plk2 can be found at GENBANK® Accession Nos. NP_{—}006613 as available Apr. 22, 2008. An exemplary amino acid sequence of human Plk3 can be found at GENBANK® Accession Nos. NP_{—}004064 as available Apr. 22, 2008. An exemplary amino acid sequence of human Plk4 can be found at GENBANK® Accession Nos. NP_{—}055079 as available Apr. 22, 2008. The sequences of Plk2, Plk3 and Plk 4 are incorporated herein by reference.

Polo-box: A distinct region of homology in the C-terminal non-catalytic domain of a polo-like kinase. This domain plays an essential role in subcellular localization of these kinases. The PBD is composed of two structurally-similar motifs, PB1 and PB2, that form a phospho-peptide-binding module by interacting with each other. The core sequence of the PB1 (corresponding to residues 513 through 542 of Cdc5 and residues 410 through 439 of mammalian Plk) is as follows:

KWVDYSX₁KX₂GX₃X₄YQLX₅X₆X₇X₈X₉X₁₀VX₁₁FN (SEQ ID NO: 11), wherein X₁, X₂, X₃, X₄, X₅, X₆, X₇, X₈, X₉, X₁₀ and X₁₁, can be any amino acid.

The PB2 motif of Plk1 bears the His538 and Lys540 residues that have electrostatic interactions with a phosphorylated serine/threonine group. The specified residues in this sequence are highly conserved across members of the polo-like kinase family of proteins. In particular, as discussed herein and by Lee et al. (PNAS, USA. 95:9301-9306, 1998), mutation of the second residue (a tryptophan) to a phenylalanine essentially completely disrupts subcellular localization of polo-like kinase to cytokinesis-related structures, and can cause a defect in mitotic functions of the polo-like kinase. In addition, mutations of His538 and Trp540 disrupt the phospho-peptide binding and thereby induces Plk1 delocalization (Elia et al., Cell 115:83-95, 2003).

Purified: The term “purified” does not require absolute purity; rather, it is intended as a relative term. Thus, for example, a purified protein preparation is one in which the protein referred to is more pure than the protein in its natural environment within a cell or within a production reaction chamber (as appropriate).

Quantitating: Determining or measuring a quantity (such as a relative quantity) of a molecule or the activity of a molecule, such as the quantity of a kinase activity of Plk1 present in a sample.

Sample: A sample, such as a biological sample, is a sample that includes biological materials (such as nucleic acid and proteins, for example Plk1). In some examples, a biological sample is obtained from an organism or a part thereof, such as an animal. In particular embodiments, the biological sample is obtained from an animal subject, such as a human subject. A biological sample can be any solid or fluid sample obtained from, excreted by or secreted by any living organism, including without limitation multicellular organisms (such as animals, including samples from a healthy or apparently healthy human subject or a human patient affected by a condition or disease to be diagnosed or investigated, such as cancer). For example, a biological sample can be a biological fluid obtained from, for example, blood, plasma, serum, urine, bile, ascites, saliva, cerebrospinal fluid, aqueous or vitreous humor, or any bodily secretion, a transudate, an exudate (for example, fluid obtained from an abscess or any other site of infection or inflammation), or fluid obtained from a joint (for example, a normal joint or a joint affected by disease, such as a rheumatoid arthritis, osteoarthritis, gout or septic arthritis). A biological sample can also be a sample obtained from any organ or tissue (including a biopsy or autopsy specimen, such as a tumor biopsy) or can include a cell (whether a primary cell or cultured cell) or medium conditioned by any cell, tissue or organ. In some examples, a biological sample is a cell lysate, for example a cell lysate obtained from the tumor of a subject.

Sequence identity: The similarity between two nucleic acid sequences, or two amino acid sequences, is expressed in terms of the similarity between the sequences, otherwise referred to as sequence identity. Sequence identity is frequently measured in terms of percentage identity (or similarity or homology); the higher the percentage, the more similar the two sequences are.

Methods of alignment of sequences for comparison are well known in the art. Various programs and alignment algorithms are described in: Smith & Waterman Adv. Appl. Math. 2: 482, 1981; Needleman & Wunsch J. Mol. Biol. 48: 443, 1970; Pearson & Lipman Proc. Natl. Acad. Sci. USA 85: 2444, 1988; Higgins & Sharp Gene 73: 237-244, 1988; Higgins & Sharp CABIOS 5: 151-153, 1989; Corpet et al. Nuc. Acids Res. 16, 10881-90, 1988; Huang et al. Computer Appls. in the Biosciences 8, 155-65, 1992; and Pearson et al. Meth. Mol. Bio. 24, 307-31, 1994. Altschul et al. (J. Mol. Biol. 215:403-410, 1990), presents a detailed consideration of sequence alignment methods and homology calculations.

The NCBI Basic Local Alignment Search Tool (BLAST) (Altschul et al. J. Mol. Biol. 215:403-410, 1990) is available from several sources, including the National Center for Biotechnology Information (NCBI, Bethesda, Md.) and on the Internet, for use in connection with the sequence analysis programs blastp, blastn, blastx, tblastn and tblastx.

Specific binding agent: An agent that binds substantially only to a defined target. Thus, a Plk1 protein-specific binding agent binds substantially to only the polo-like kinase Plk1. As used herein, a Plk1 specific binding agent includes anti-Plk1 antibodies (and functional fragments thereof) and other agents (such as peptides) that bind substantially only to Plk1. For instance, a Plk1-specific binding agent would bind substantially only to Plk1. In other examples, a specific binding agent is a binding agent that binds Plk1 substrate, such as the Plk1 substrate peptides disclosed herein. In some examples, a Plk1 substrate peptide-binding agent is isolated Plk1, which binds to phosphorylated Plk1 substrates. In some examples, a Plk1 substrate binding agent is an antibody that binds the Plk1 substrate, such as the peptides disclosed herein, when the Plk1 peptide substrate is phosphorylated. In some examples, a Plk1 substrate peptide binding agent is an antibody that binds the Plk1 substrate, such as the peptides disclosed herein, when the Plk1 peptide substrate is not phosphorylated.

Substrate: A molecule that is acted upon by an enzyme. A substrate binds with the enzyme's active site, and an enzyme-substrate complex is formed. In some examples, the enzyme catalyses the incorporation of an atom or other molecule into the substrate, for example a kinase can incorporate a phosphate into the substrate, such as a peptide, thus forming a phospho-substrate. In some examples, the kinase Plk1 phosphorylates substrate peptides (such as the peptides disclosed herein) on threonine residues within the peptide.

Test agent: Any agent that is tested for its effects, for example its effects on the kinase activity of a kinase, such as the kinase activity of Plk1. In some embodiments, a test agent is a chemical compound, such as a chemotherapeutic agent or even an agent with unknown biological properties.

Transformed: A transformed cell is a cell into which has been introduced a nucleic acid molecule by molecular biology techniques. As used herein, the term transformation encompasses all techniques by which a nucleic acid molecule might be introduced into such a cell, including transfection with viral vectors, transformation with plasmid vectors, and introduction of DNA by electroporation, lipofection, and particle gun acceleration.

Vector: A nucleic acid molecule as introduced into a host cell, thereby producing a transformed host cell. Recombinant DNA vectors are vectors having recombinant DNA. A vector can include nucleic acid sequences that permit it to replicate in a host cell, such as an origin of replication. A vector can also include one or more selectable marker genes and other genetic elements known in the art. Viral vectors are recombinant DNA vectors having at least some nucleic acid sequences derived from one or more viruses.

### II. Description of Several Embodiments

Over expression of Polo-Like Kinase 1 (Plk1) can be a diagnostic marker for many types of malignancies, such as non-small-cell lung cancer, oropharyngeal carcinoma, esophageal carcinoma, melanoma, colorectal cancer, hepatoblastoma, and non-Hodgkin lymphoma. However, the conventional determination of Plk1 expression levels and kinase activity is laborious and time consuming, typically relying on immunoblot assays and immunocomplex assays using the general kinase substrate casein. Such assays can also suffer from low sensitivity and specificity for the activity of Plk1. Thus, the need exists for more sensitive and specific assays for Plk1 activity. Disclosed herein is a Plk1 kinase assay that is highly specific for Plk1, in that the assay can specifically measure the kinase activity of Plk1, without interference from other kinases that may be present in the sample to be tested. As disclosed herein, this assay is based on the development of unique peptide substrates that exhibit high selectivity and sensitivity for Plk1 kinase activity (thus allowing small samples to be analyzed) while maintaining a high degree of specificity for Plk1. The assays can be used to detect Plk1 activity in a biological cell sample, such as a biopsy sample. As Plk1 has been associated with the presence of tumors (and the prognosis for a subject with a tumor), these assays can be used to identify a tumor in a subject foretell tumorigenesis, and/or determine the prognosis of the subject, such as a subject suffering from cancer. The disclosed assays are also of use in identifying for Plk1 inhibitors, which can be used to treat cancer or alternatively as lead compounds for the development of anti-cancer therapeutics.

### A. Plk1 Substrate Polypeptides

Disclosed herein are isolated peptide substrates of Plk1 that are specific for Plk1, in that that they bind and are phosphorylated by Plk1, but not by the related polo kinases Plk2, Plk3 and Plk4. The disclosed polypeptides include a portion of the amino acid sequence of PBIP1 (a natural substrate of Plk1), but are optimized to provided a peptide sequence with enhanced specificity and sensitivity over the native PBIP1 sequence. The disclosed polypeptides include a portion of the amino acid sequence of PBIP1 (a natural substrate of Plk1), but are optimized to provided a peptide sequence with enhanced specificity and sensitivity over the native PBIP1 sequence. The disclosed substrate peptides include a portion of the T78 region of PBIP, which is amino acid residues 64-85 of PBIP (residues 64-85 of SEQ ID NO: 10) and the site of phosphorylation (T78). Because the disclosed polypeptides can be phosphorylated by Plk1 exclusive of other kinases, the polypeptides are specific for Plk1 and are of use in detecting the kinase activity of Plk1 in a sample, such as a biological sample.

The disclosed polypeptides include two to ten consecutive repeats (such as 2, 3, 4, 5, 6, 7, 8, 9 or 10 repeats) of the amino acid sequence set forth as The disclosed polypeptides include two to ten consecutive repeats of the amino acid sequence set forth as X₁X₂AX₃X₄X₅PLHSTX₆X₇X₈X₉X₁₀X₁₁X₁₂ (SEQ ID NO: 1), in which within each repeat X₁ is independently any amino acid or no amino acid, X₂ is independently any amino acid or no amino acid, X₃ is independently any amino acid, X₄ is independently any amino acid, X₅ is independently any amino acid, X₆ is independently any amino acid, X₇ is independently any amino acid, X₈ is independently any amino acid or no amino acid, X₉ is independently any amino acid or no amino acid, X₁₀ is independently any amino acid or no amino acid, X₁₁ is independently any amino acid or no amino acid, and X₁₂ is independently any amino acid or no amino acid. The two to ten consecutive repeats of SEQ ID NO: 1 can be joined by a peptide linker between two and ten amino acids in length.

The disclosed peptides are about 23 amino acids in length to about 250 amino acids in length (or even greater than 250 amino acids in length, for example when part of a larger fusion protein), such as about 23, as about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100, about 101, about 102, about 103, about 104, about 105, about 106, about 107, about 108, about 109, about 110, about 111, about 112, about 113, about 114, about 115, about 116, about 117, about 118, about 119, about 120, about 121, about 122, about 123, about 124, about 125, about 126, about 127, about 128, about 129, about 130, about 131, about 132, about 133, about 134, about 135, about 136, about 137, about 138, about 139, about 140, about 141, about 142, about 143, about 144, about 145, about 146, about 147, about 148, about 149, about 150, about 151, about 152, about 153, about 154, about 155, about 156, about 157, about 158, about 159, about 160, about 161, about 162, about 163, about 164, about 165, about 166, about 167, about 168, about 169, about 170, about 171, about 172, about 173, about 174, about 175, about 176, about 177, about 178, about 179, about 180, about 181, about 182, about 183, about 184, about 185, about 186, about 187, about 188, about 189, about 190, about 191, about 192, about 193, about 194, about 195, about 196, about 197, about 198, about 199, about 200, about 201, about 202, about 203, about 204, about 205, about 206, about 207, about 208, about 209, about 210, about 211, about 212, about 213, about 214, about 215, about 216, about 217, about 218, about 219, about 220, about 221, about 222, about 223, about 224, about 225, about 226, about 227, about 228, about 229, about 230, about 231, about 232, about 233, about 234, about 235, about 236, about 237, about 238, about 239, about 240, about 241, about 242, about 243, about 244, about 245, about 246, about 247, about 248, about 249, or about 250 amino acids in length, for example about 40 to about 69, about 46 to about 92, about 69 to about 115, about 92 to about 138, about 115 to about 161, about 138 to about 184, about 161 to about 207, about 184 to about 230 amino acid or about 207 to 250 amino acids in length or greater. In this context, it is understood that “about” refers to an integer quantity.

In some embodiments, the Plk1 substrate peptide includes the amino acid sequence set forth as SEQ ID NO: 1, wherein X₁ is independently any amino acid or no amino acid, X₂ is independently any amino acid or no amino acid, X₃ is phenylalanine, X₄ is aspartic acid, X₅ is proline, X₆ is independently any amino acid, X₇ is independently any amino acid, X₈ is independently any amino acid or no amino acid, X₉ is independently any amino acid or no amino acid, X₁₀ is independently any amino acid or no amino acid, X₁₁ is independently any amino acid or no amino acid, and X₁₂ is independently any amino acid or no amino acid. For example, the disclosed Plk1 substrate peptides can include two to ten repeats (such as 2, 3, 4, 5, 6, 7, 8, 9 or 10 repeats) of the amino acid sequence set forth as X₁X₂AFDPPLHSTX₆X₇X₈X₉X₁₀X₁₁X₁₂ (SEQ ID NO: 2)

In some embodiments, the Plk1 substrate peptide includes the amino acid sequence set forth as SEQ ID NO: 1, wherein X₁ is independently any amino acid or no amino acid, X₂ is independently any amino acid or no amino acid, X₃ is phenylalanine, X₄ is aspartic acid, X₅ is proline, X₆ is alanine, X₇ isoluecine, X₈ is independently any amino acid or no amino acid, X₉ is independently any amino acid or no amino acid, X₁₀ is independently any amino acid or no amino acid, X₁₁ is independently any amino acid or no amino acid, and X₁₂ is independently any amino acid or no amino acid. For example, the disclosed Plk1 substrate peptides can include two to ten repeats (such as 2, 3, 4, 5, 6, 7, 8, 9 or 10 repeats) of the amino acid sequence set forth as X₁X₂AFDPPLHSTAIX₈X₉X₁₀X₁₁X₁₂ (SEQ ID NO: 3)

In some embodiments, the Plk1 substrate peptide includes the amino acid sequence set forth as SEQ ID NO: 1, wherein X₁ is tyrosine, X₂ is glutamic acid, X₃ is phenylalanine, X₄ is aspartic acid, X₅ is proline, X₆ is alanine, X₇ is isoluecine, X₈ is tyrosine, X₉ is alanine, X₁₀ is aspartic acid, X₁₁ is glutamic acid, and X₁₂ is glutamic acid. For example, the disclosed Plk1 substrate peptides can include two to ten repeats (such as 2, 3, 4, 5, 6, 7, 8, 9 or 10 repeats) of the amino acid sequence set forth as YEAFDPPLHSTAIYADEE (SEQ ID NO: 4).

In some embodiments, the Plk1 substrate peptide includes the amino acid sequence set forth as SEQ ID NO: 1, wherein X₁ is phenylalanine, X₂ is glutamic acid, X₃ is phenylalanine, X₄ is aspartic acid, X₅ is proline, X₆ is alanine, X₇ is isoluecine, X₈ is phenylalanine, X₉ is alanine, X₁₀ is aspartic acid, X₁₁ is glutamic acid, and X₁₂ is glutamic acid. For example, the disclosed Plk1 substrate peptides can include two to ten repeats (such as 2, 3, 4, 5, 6, 7, 8, 9 or 10 repeats) of the amino acid sequence set forth as FEAFDPPLHSTAIFADEE (SEQ ID NO: 5).

In some examples, the disclosed peptides include two to ten consecutive repeats (such as 2, 3, 4, 5, 6, 7, 8, 9 or 10 repeats) of the amino acid sequence set forth as SEQ ID NO: 4 and/or SEQ ID NO: 5. For example, the peptide can include SEQ ID NO: 4 and SEQ ID NO: 5. However, the total number of repeats of SEQ ID NO: 1 is from two to ten (such as 2, 3, 4, 5, 6, 7, 8, 9 or 10).

The Plk1 substrate peptides that include two to ten consecutive repeats of SEQ ID NO: 1 can be joined by a peptide linker that is between two and ten amino acids in length, such as about 2, about 3, about 4, about 5, about 6, about 7, about 8, about 9 or about 10 amino acids in length. Depending on such factors as the molecules to be linked, and the conditions in which the method of detection is performed, the linker can vary in length and composition for optimizing such properties as flexibility, and stability. The linker is a peptide heterologous to the Plk1 substrate peptides. In some examples, a linker is peptide such as poly-lysine, poly-glutamine, poly-glycine, poly-proline or any combination combinations thereof. In specific examples, a peptide linker is the amino acid sequence GGPGG (SEQ ID NO: 12). In some examples, the peptide linker can be designed to be either hydrophilic or hydrophobic in order to enhance the desired binding characteristics of Plk1 substrate peptide and Plk1, an antibody that specifically binds the Plk1 substrate peptide when the peptide is not phosphorylated or an antibody that specifically binds the Plk1 substrate peptide when the peptide is phosphorylated on one or more threonine residues. The peptide linker and the individual units of the Plk1 substrate peptide (such as the individual units set forth as SEQ ID NO: 1) can be encoded as a single fusion polypeptide such that the peptide linker and the individual units of the Plk1 substrate peptide are joined by peptide bonds.

In some examples, the Plk1 substrate peptides include four consecutive repeats (for example four consecutive repeats of SEQ ID NO: 4 and/or SEQ ID NO: 5) of the amino acid sequence according to SEQ ID NO: 1 linked by the peptide linker GGPGG (SEQ ID NO: 12), wherein within each repeat X₁ is independently phenylalanine or tyrosine, X₂ is glutamic acid, X₃ is phenylalanine, X₄ is aspartic acid, X₅ is proline, X₆ is alanine, X₇ is isoluecine, X₈ is independently phenylalanine or tyrosine, X₉ is alanine, X₁₀ is aspartic acid, X₁₁ is glutamic acid, and X₁₂ is glutamic acid. This peptide can include the amino acid sequence set forth as X₁EAFDPPLHSTAIX₂ADEEGGPGGX₃EAFDPPLHSTAIX₄ADEEGGPGG X₅EAFDPPLHSTAIX₆ADEEGGPGGX₇EAFDPPLHSTAIX₈ADEE (SEQ ID NO: 6), wherein X₁ is tyrosine or phenylalanine, X₂ is tyrosine or phenylalanine, X₃ is tyrosine or phenylalanine, X₄ is tyrosine or phenylalanine, X₅ is tyrosine or phenylalanine, X₆ is tyrosine or phenylalanine, X₇ is tyrosine or phenylalanine, and X₈ is tyrosine or phenylalanine.

In some examples, the Plk1 substrate peptides include six consecutive repeats of the amino acid sequence according to SEQ ID NO: 1 (for example six consecutive repeats of SEQ ID NO: 4 and/or SEQ ID NO: 5), wherein within each repeat X₁ is independently phenylalanine or tyrosine, X₂ is glutamic acid, X₃ is phenylalanine, X₄ is aspartic acid, X₅ is proline, X₆ is alanine, X₇ is isoluecine, X₈ is independently phenylalanine or tyrosine, X₉ is alanine, X₁₀ is aspartic acid, X₁₁ is glutamic acid, and X₁₂ is glutamic acid. This peptide can include the amino acid sequence set forth as

X₁EAFDPPLHSTAIX₂ADEEGGPGGX₃EAFDPPLHSTAIX₄ADEEGGPGG X₅EAFDPPLHSTAIX₆ADEEGGPGGX₇EAFDPPLHSTAIX₈ADEEGGPGG X₉EAFDPPLHSTAIX₁₀ADEEGGPGGX₁₁EAFDPPLHSTAIX₁₂ADEE (SEQ ID NO: 7), wherein X₁ is tyrosine or phenylalanine, X₂ tyrosine or phenylalanine, X₃ is tyrosine or phenylalanine, X₄ is tyrosine or phenylalanine, X₅ is tyrosine or phenylalanine, X₆ is tyrosine or phenylalanine, X₇ is tyrosine or phenylalanine, X₈ is tyrosine or phenylalanine, X₉ is tyrosine or phenylalanine, X₁₀ is tyrosine or phenylalanine, X₁₁ is tyrosine or phenylalanine and X₁₂ is tyrosine or phenylalanine.

In some examples, the Plk1 substrate peptides include six consecutive repeats of the amino acid sequence according to SEQ ID NO:1, wherein within each repeat X₁ is phenylalanine, X₂ is glutamic acid, X₃ is phenylalanine, X₄ is aspartic acid, X₅ is proline, X₆ is alanine, X₇ is isoluecine, X₈ is phenylalanine, X₉ is alanine, X₁₀ is aspartic acid, X₁₁ is glutamic acid, and X₁₂ is glutamic acid. This peptide can include the amino acid sequence set forth as

### GGPGGFEAFDPPLHSTAIFADEEGGPGGFEAFDPPLHSTAIFADEEGGPGGFE AFDPPLHSTAIFADEEGGPGGFEAFDPPLHSTAIFADEEGGPGGFEAFDPPLHS TAIFADEEGGPGGFEAFDPPLHSTAIFADEE (SEQ ID NO: 8).

In some examples, the Plk1 substrate peptides include eight consecutive repeats of the amino acid sequence according to SEQ ID NO: 1 (for example eight consecutive repeats of SEQ ID NO: 4 and/or SEQ ID NO: 5), wherein within each repeat X₁ is independently phenylalanine or tyrosine, X₂ is glutamic acid, X₃ is phenylalanine, X₄ is aspartic acid, X₅ is proline, X₆ is alanine, X₇ is isoluecine, X₈ is independently phenylalanine or tyrosine X₉ is alanine, X₁₀ is aspartic acid, X₁₁ is glutamic acid, and X₁₂ is glutamic acid. This peptide can the amino acid sequence set forth as

X₁EAFDPPLHSTAIX₂ADEEGGPGGX₃EAFDPPLHSTAIX₄ADEEGGPGG X₅EAFDPPLHSTAIX₆ADEEGGPGGX₇EAFDPPLHSTAIX₈ADEEGGPGG X₉EAFDPPLHSTAIX₁₀ADEEGGPGGX₁₁EAFDPPLHSTAIX₁₂ADEEGGPGG X₁₃EAFDPPLHSTAIX₁₄ADEEGGPGGX₁₅EAFDPPLHSTAIX₁₆ADEE (SEQ ID NO: 9), wherein X₁ is tyrosine or phenylalanine, X₂ is tyrosine or phenylalanine, X₃ is tyrosine or phenylalanine, X₄ is tyrosine or phenylalanine, X₅ is tyrosine or phenylalanine, X₆ is tyrosine or phenylalanine, X₇ is tyrosine or phenylalanine, X₈ is tyrosine or phenylalanine, X₉ is tyrosine or phenylalanine, X₁₀ is tyrosine or phenylalanine, X₁₁ is tyrosine or phenylalanine, X₁₂ is tyrosine or phenylalanine, X₁₃ is tyrosine or phenylalanine, X₁₄ is tyrosine or phenylalanine, X₁₅ is tyrosine or phenylalanine and X₁₆ is tyrosine or phenylalanine.

In some embodiments, the Plk1 substrate peptide includes an additional heterologous amino acid sequence, for example a glutathione-S-transferase (GST), biotin, avidin or streptavidin heterologous amino acid sequence. The inclusion of a heterologous peptide fusion partner (such as GST, biotin, avidin or streptavidin), can aid in attachment of the peptide to a solid surface and/or purification of the peptides disclosed herein. The heterologous fusion proteins can be constructed such that the amino acid sequence of the heterologous peptide or peptide fusion partner is fused either/or N-terminally or C-terminally to the Plk1 kinase substrate peptides disclosed herein. The disclosed peptides may be modified by a variety of chemical techniques to produce derivatives having essentially the same activity (a substrate of Plk1 kinase) as the unmodified proteins, and optionally having other desirable properties, for example to attached them to a solid support, such as a microtiter plate (for example a multiwell plate, such as a 96 well or 384 well plate), bead (for example an agarose bead) and the like. In practice, microtiter plates may conveniently be utilized as the solid phase. The surfaces may be prepared in advance, stored, and shipped to another location(s).

In some embodiments, the disclosed peptide substrates of Plk1 are detectably labeled, for example with fluorophore (for example FTIC, PE, a fluorescent protein, and the like), an enzyme (for example HRP), a radiolabel, or a nanoparticle (for example a gold particle or a semiconductor nanocrystal, such as a quantum dot (QDOT®)). Methods for labeling are discussed for example Ausubel et al. (In Current Protocols in Molecular Biology, John Wiley & Sons, New York, 1998).

### B. Nucleic Acids Encoding Plk1 Substrate Polypeptides

The present disclosure also concerns nucleic acid constructs including polynucleotide sequences that encode the peptide substrates of Plk1 disclosed herein, such as isolated nucleic acid molecules and vectors including such nucleic acid molecules. These polynucleotides include DNA, cDNA and RNA sequences, which encode the polypeptide of interest. Thus, this disclosure encompasses the polynucleotides, encoding the amino acid sequences comprising two to ten repeats (such as 2, 3, 4, 5, 6, 7, 8, 9 or 10 repeats) of the disclosed polypeptides include two to ten consecutive repeats (such as 2, 3, 4, 5, 6, 7, 8, 9 or 10 repeats) of the amino acid sequence set forth as X₁X₂AX₃X₄X₅PLHSTX₆X₇X₈X₉X₁₀X₁₁X₁₂ (SEQ ID NO: 1), in which within each repeat X₁ is independently any amino acid or no amino acid, X₂ is independently any amino acid or no amino acid, X₃ is independently any amino acid, X₄ is independently any amino acid, X₅ is independently any amino acid, X₆ is independently any amino acid, X₇ is independently any amino acid, X₈ is independently any amino acid or no amino acid, X₉ is independently any amino acid or no amino acid, X₁₀ is independently any amino acid or no amino acid, X₁₁ is independently any amino acid or no amino acid, and X₁₂ is independently any amino acid or no amino acid. The two to ten consecutive repeats of SEQ ID NO: 1 can be joined by a peptide linker between two and ten amino acids in length. These peptides are described above.

The nucleic acid constructs can include polynucleotides that encode heterologous polypeptides in addition to those set forth as SEQ ID NO: 1, for example peptide that include peptide linkers (such as set forth as SEQ ID NO: 12) or other moieties to aid in the purification, detection (such as heterologous fluorescent protein sequences, such as green fluorescent protein and the like), and/or attachment of the peptides to a solid surface (such as GST, biotin, avidin or streptavidin).

The coding region may be altered by taking advantage of the degeneracy of the genetic code to alter the coding sequence such that, while the nucleotide sequence is altered, it nevertheless encodes a peptide having an amino acid sequence the same as the disclosed peptide sequences, for example for optimization of expression in a host cell, such as a bacterial host cell, such as E. coli. Based upon the degeneracy of the genetic code, variant DNA molecules may be derived from encoding sequences disclosed herein using standard DNA mutagenesis techniques as described above, or by synthesis of DNA sequences.

To produce such nucleic acid constructs, polynucleotide sequences encoding peptides are inserted into a suitable expression vector, such as a plasmid expression vector. Procedures for producing polynucleotide sequences encoding the peptides disclosed herein and for manipulating them in vitro are well known to those of skill in the art, and can be found (see for example, Sambrook et al., Molecular Cloning, a Laboratory Manual, 2nd edition, Cold Spring Harbor Press, Cold Spring Harbor, N.Y., 1989, and Ausubel et al., Current Protocols in Molecular Biology, Greene Publishing Associates and John Wiley & Sons, New York, N.Y., 1994).

A wide variety of cloning and in vitro amplification methodologies are well known to persons skilled in the art. A nucleic acid encoding a polypeptide can be cloned or amplified by in vitro methods, such as the polymerase chain reaction (PCR), the ligase chain reaction (LCR), the transcription-based amplification system (TAS), the self-sustained sequence replication system (3SR) and the Qβ replicase amplification system (QB). Methods for the manipulation and insertion of the nucleic acids of this disclosure into vectors are well known in the art (see for example, Sambrook et al., Molecular Cloning, a Laboratory Manual, 2nd edition, Cold Spring Harbor Press, Cold Spring Harbor, N.Y., 1989, and Ausubel et al., Current Protocols in Molecular Biology, Greene Publishing Associates and John Wiley & Sons, New York, N.Y., 1994). PCR methods are described in, for example, U.S. Pat. No. 4,683,195; Mullis et al., Cold Spring Harbor Symp. Quant. Biol. 51:263, 1987; and Erlich, ed., PCR Technology, (Stockton Press, NY, 1989).

A polynucleotide sequence encoding the disclosed peptides can be operatively linked to expression control sequences. An expression control sequence operatively linked to a coding sequence is ligated such that expression of the coding sequence is achieved under conditions compatible with the expression control sequences. The expression control sequences include, but are not limited to, appropriate promoters, enhancers, transcription terminators, a start codon (i.e., ATG) in front of a protein-encoding gene, splicing signal for introns, maintenance of the correct reading frame of that gene to permit proper translation of mRNA, and stop codons. A promoter is an array of nucleic acid control sequences that directs transcription of a nucleic acid. A promoter includes necessary nucleic acid sequences (which can be) near the start site of transcription, such as in the case of a polymerase II type promoter (a TATA element). A promoter also can include distal enhancer or repressor elements, which can be located as much as several thousand base pairs from the start site of transcription. Both constitutive and inducible promoters are included (see, for example, Bitter et al., Methods in Enzymology 153:516-544, 1987).

The polynucleotides include a recombinant DNA can be incorporated into a vector into an autonomously replicating plasmid or virus or into the genomic DNA of a prokaryote or eukaryote, or which exists as a separate molecule (for example a cDNA) independent of other sequences. Typically, the nucleic acid constructs encoding the peptides of this disclosure are plasmids. However, other vectors (for example, viral vectors, phage, cosmids, etc.) can be utilized to replicate the nucleic acids. In the context of this disclosure, the nucleic acid constructs typically are expression vectors that contain a promoter sequence, which facilitates the efficient transcription of the inserted genetic sequence of the host. The expression vector typically contains an origin of replication, a promoter, as well as specific nucleic acid sequences that allow phenotypic selection of the transformed cells.

DNA sequences encoding the peptides of this disclosure can be expressed in vitro by DNA transfer into a suitable host cell. Thus, also disclosed are host cells that include vectors encoding the peptides of this disclosure. The cell may be prokaryotic or eukaryotic. The term also includes any progeny of the subject host cell. It is understood that all progeny may not be identical to the parental cell since there may be mutations that occur during replication. Methods of stable transfer, meaning that the foreign DNA is continuously maintained in the host, are known in the art.

Transformation of a host cell with recombinant DNA can be carried out by conventional techniques as are well known to those skilled in the art. Where the host is prokaryotic, such as E. coli, competent cells, which are capable of DNA uptake can be prepared from cells harvested after exponential growth phase and subsequently treated by the CaCl₂ method using procedures well known in the art. Alternatively, MgCl₂ or RbCl can be used. Transformation can also be performed after forming a protoplast of the host cell if desired, or by electroporation.

When the host is a eukaryote, such methods of transfection of DNA as calcium phosphate coprecipitates, conventional mechanical procedures such as microinjection, electroporation, insertion of a plasmid encased in liposomes, or virus vectors can be used. Eukaryotic cells can also be co-transformed with polynucleotide sequences encoding the polypeptide of interest, and a second foreign DNA molecule encoding a selectable phenotype, such as the herpes simplex thymidine kinase gene. Another method is to use a eukaryotic viral vector, such as simian virus 40 (SV40) or bovine papilloma virus, to transiently infect or transform eukaryotic cells and express the protein (see for example, Eukaryotic Viral Vectors, Cold Spring Harbor Laboratory, Gluzman ed., 1982).

### C. Assays for Detecting Plk1 and Plk1 Kinase Activity

This disclosure also relates to methods for detecting Plk1 kinase activity in a sample, such as a biological sample, for example, a sample obtained from a subject, such as subject of interest, for example a subject with a tumor.

Appropriate samples for use in the methods disclosed herein include any conventional biological sample for which information about Plk1 activity is desired. Samples include those obtained from, excreted by or secreted by any living organism, such as eukaryotic organisms including without limitation, multicellular organisms (such as animals, including samples from a healthy or apparently healthy human subject or a human patient affected by a condition or disease to be diagnosed or investigated, such as cancer), clinical samples obtained from a human or veterinary subject, for instance blood or blood-fractions, biopsied tissue. Standard techniques for acquisition of such samples are available. See, for example Schluger et al., J. Exp. Med. 176:1327-33 (1992); Bigby et al., Am. Rev. Respir. Dis. 133:515-18 (1986); Kovacs et al., NEJM 318:589-93 (1988); and Ognibene et al., Am. Rev. Respir. Dis. 129:929-32 (1984). Biological samples can be obtained from any organ or tissue (including a biopsy or autopsy specimen, such as a tumor biopsy) or can comprise a cell (whether a primary cell or cultured cell) or medium conditioned by any cell, tissue or organ. In some embodiments, a biological sample is a cell lysate, such as a cell lysate from cells of a tumor, such as a tumor of a subject diagnosed with cancer. Cell lysate contains many of the proteins contained in a cell, and includes for example Plk1. Methods for obtaining a cell lysate are well known in the art and can be found for example in Ausubel et al. (In Current Protocols in Molecular Biology, John Wiley & Sons, New York, 1998). In some example, the sample is obtained from a subject diagnosed as having breast cancer, ovarian cancer, non-small cell lung cancer, head/neck cancer, colon cancer, endometrial cancer, esophageal carcinoma, or leukemia.

The disclosed methods for detecting the kinase activity of Plk1 (and thus the presence of Plk1) are based on the discovery that a peptide can be designed as highly specific substrate of Plk1 that has high sensitivity for Plk1 activity, such as the peptides described above. In several embodiments, the disclosed methods include contacting a sample, such as a biological sample, with a disclosed peptide substrate of Plk1, such as the peptides disclosed above, in the presence of adenosine triphosphate, or an analog thereof, for a period of time sufficient for the Plk1 kinase to transfer a phosphate from the adenosine triphosphate, or analog thereof to the Plk1 substrate peptides, if Plk1 is present in the sample. The presence and/or amount of the phosphorylated peptide is detected, thereby detecting and/or quantitating Plk1 kinase activity in the sample. The presence of a phosphorylated peptide can be determined using any method known to one of skill in the art.

In several examples, the presence of the phosphorylated peptide is determined using an antibody that specifically binds the polypeptide, such as a monoclonal or polyclonal antibody. The presence of antibody:antigen complexes can be determined using methods known in the art. For example, the antibody can include a detectable label, such as a fluorophore, radiolabel, or enzyme, which permits detection of the antibody, for example using enzyme-linked immunosorbent assay (ELISA).

An ELISA is a biochemical technique that can be used mainly to detect the presence of an antibody or an antigen in a sample, for example an antibody that specifically binds a phosphorylated peptide, such as a phosphorylated Plk1 substrate peptide disclosed herein. In some embodiments, an amount of antigen, such as a Plk1 substrate disclosed herein is affixed to a surface, a sample is passed over the Plk1 substrate to determine if the sample has Pkl activity as evidenced by phosphorylation of the PLK1 substrate by a sample containing Plk1. A specific antibody that specifically binds a phosphorylated Plk1 substrate, or in some examples a known amount of Plk1 enzyme, is washed over the surface so that it can bind to the phosphorylated Plk1 substrate. In some examples, the antibody can be linked to an enzyme, for example directly conjugated or through a secondary antibody, and a substance is added that the enzyme can convert to a detectable signal. Thus, in the case of fluorescence ELISA, when light of the appropriate wavelength is shone upon the sample, any antigen:antibody complexes will fluoresce so that the amount of antigen in the sample can be inferred through the magnitude of the fluorescence. The antigen is usually immobilized on a solid support (for example polystyrene microtiter plate) either non-specifically (for example via adsorption to the surface) or specifically (for example via capture by another antibody specific to the same antigen, in a “sandwich” ELISA). Between each step the plate is typically washed with a mild detergent solution, such as phospho-buffered saline with or without NP40 or TWEEN© to remove any proteins or antibodies that are not specifically bound. After the final wash step the plate is developed by adding an enzymatic substrate to produce a visible signal, which indicates the quantity of antigen in the sample.

In some embodiments, the presence of the phosphorylated peptide substrate of Plk1 is detected by contacting the sample with a specific binding agent (which can be detectably labeled, for example with a fluorophore (for example FTIC, PE, a fluorescent protein, and the like), an enzyme (for example HRP), a radiolabel, or a nanoparticle (for example a gold particle or a semiconductor nanocrystal, such as a quantum dot (QDOT®)) that specifically binds the peptide when the peptide is phosphorylated on one or more threonine residues, such that the specific binding agent does not specifically bind the peptide when the peptide is not phosphorylated. The presence of a complex formed between the specific binding agent and the peptide is detected, thereby detecting the kinase activity of Plk1 in the sample. In some embodiments, the specific binding agent is an antibody that specifically binds to the peptide when the peptide is phosphorylated on a threonine residue. In other embodiments, the specific binding agent is isolated Plk1, which binds to the Plk1 substrate peptides when they are phosphorylated on threonine residues. In some embodiments, the specific binding agent is detectably labeled. Thus, in some embodiments the specific binding agent is a detectably labeled antibody that specifically binds to the Plk1 substrate peptide when the Plk1 substrate peptide is phosphorylated on a threonine residue. In other embodiments, the specific binding agent is detectably labeled isolated Plk1 that specifically binds to the Plk1 substrate peptide when the Plk1 substrate peptide is phosphorylated on a threonine residue. In some examples, the method can include contacting the sample with a second specific binding agent that specifically binds to the specific binding agent. In some examples, the second specific binding agent is detectably labeled, for example with a fluorophore (such as FTIC, PE, a fluorescent protein, and the like), an enzyme (such as HRP), a radiolabel, or a nanoparticle (such as a gold particle or a semiconductor nanocrystal, such as a quantum dot (QDOT®)). In some examples, the second specific binding agent is an antibody, such as a second antibody that specifically binds to the antibody that specifically binds to the Plk1 substrate peptide when the peptide is phosphorylated on a threonine residue, or an antibody that specifically binds Plk1.

Detection can be conducted in a liquid phase. In some examples, the peptide and the specific binding agent that binds the peptide are used that are tagged with different detectable labels, such as a first and second tag. In one example, the first and second tag interact when in proximity, such as when the peptide and the specific binding agent that binds the peptide are in a complex. The relative proximity of the first and second tags is determined by measuring a change in the intrinsic fluorescence of the first or second tag. Commonly, the emission of the first tag is quenched by proximity of the second tag. After incubation, the presence or absence of a detectable tag emission is detected. The detected emission can be any of the following: an emission by the first tag, an emission by the second tag, and an emission resulting from a combination of the first and second tag. To detect a complex between the peptide and the specific binding agent that binds the peptide a change in the signal, due to formation of a complex between the peptide and the specific binding agent that binds the peptide, is detected (for example, as an increase in fluorescence as a result of fluorescence resonance energy transfer (FRET), as an increase in quenching that leads to an decrease in signal from either or both of the tags, a change in signal color, and the like).

Many appropriate interactive tags are known. For example, fluorescent tags, dyes, enzymatic tags, and antibody tags are all appropriate. Examples of preferred interactive fluorescent tag pairs include terbium chelate and TRITC (tetramethylrhodamine isothiocyanate), europium cryptate and allophycocyanin and many others known to one of ordinary skill in the art. Similarly, two colorimetric tags can result in combinations that yield a third color, for example, a blue emission in proximity to a yellow emission provides an observed green emission.

With regard to preferred fluorescent pairs, there are a number of fluorophores that are known to quench one another. Fluorescence quenching is a bimolecular process that reduces the fluorescence quantum yield, typically without changing the fluorescence emission spectrum. Quenching can result from transient excited state interactions, (collisional quenching) or, for example, from the formation of nonfluorescent ground state species. Self-quenching is the quenching of one fluorophore by another; it tends to occur when high concentrations, labeling densities, or proximity of tags occurs. Fluorescence resonance energy transfer (FRET) is a distance dependent excited state interaction in which emission of one fluorophore is coupled to the excitation of another that is in proximity (close enough for an observable change in emissions to occur). Some excited fluorophores interact to form excimers, which are excited state dimers that exhibit altered emission spectra (for example, phospholipid analogs with pyrene sn-2 acyl chains); see, Haugland (1996) Handbook of Fluorescent Probes and Research Chemicals, Published by Molecular Probes, Inc., Eugene, Oreg., for example at chapter 13).

In most uses, the first and second tags are different, in which case FRET can be detected by the appearance of sensitized fluorescence of the acceptor or by quenching of the donor fluorescence. When the first and second tags are the same, FRET is detected by the resulting fluorescence depolarization. In addition to quenching between fluorophores, individual fluorophores are also quenched by nitroxide-tagged molecules such as fatty acids. Spin tags such as nitroxides are also useful in the liquid phase assays describer herein. Liquid phase assays described herein can be performed in essentially any liquid phase container for example a container designed for high throughput screening such as a multiwell microtiter dish (for example, 96 well, 384 well, etc).

The phosphorylated PLK1 substrate can be detected for example using stains specific for phosphorylated proteins in gels. In some embodiments, the phosphorylated peptide is detected by measuring the transfer of a labeled phosphate (such as radioactive phosphorus (³²P)) from the γ phosphate of a nucleotide triphosphate, such as ATP to the peptide. By addition of γ phosphate labeled triphosphate, such as ATP, into sample, the presence of phosphorylated peptide can be determined. In some embodiments, a γ phosphate labeled triphosphate, such as ATP is added to the sample. Typically, the γ phosphate label is a radioisotope label, although any label that can be transferred via a kinase reaction is contemplated by this disclosure. By this methodology, the activation, degree of activation, and/or inhibition of the kinase activity of Plk1 can be determined by incorporation of ³²P into a peptide substrate of Plk1.

The amount of the phosphorylated peptide can be compared to a control. In several embodiments, the control is a known value indicative of basal phosphorylation of the peptide, for example in the absence of Plk1. In several embodiments, the control is the amount of phosphorylated peptide formed in the presence of a known amount of isolated Plk1. In other embodiments, the sample is a patient sample and the control is a patient sample from the patient at an earlier time. A difference between the amount of the phosphorylated peptide formed and a control indicates that the sample has more or less Plk1 kinase activity than a control.

In some embodiments, the difference in the amount of phosphorylated peptide relative to a control is a statistically significant difference. In some embodiments, the difference in the amount of phosphorylated peptide relative to a control is at least about 10%, such as at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, at least about 500%, or greater then 500%.

In some embodiments of the method, the peptide is attached to a solid support (for example a multiwell plate (such as a microtiter plate), bead, membrane or the like), for example through a GST moiety fused to the peptide, a streptavidin moiety fused to the peptide, an avidin moiety fused to the peptide, a biotin moiety fused to the peptide or other linker. In practice, microtiter plates may conveniently be utilized as the solid phase. The surfaces may be prepared in advance, stored, and shipped to another location(s). Linkers for attaching peptides to solid supports are well known in the art.

### D. Methods of Identifying an Inhibitor of Plk1

This disclosure also relates to methods for determining if a test agent inhibits the kinase activity of Plk1, for example to screen compounds to determine if the compounds are of use in treating cancer (or other disease associated with Plk1) by inhibiting the activity of Plk1. In one example, using the peptides and assays described herein, compounds can be screened in a high through put manner to determine if the compounds can be used to treat cancer, or should be tested in clinical trials or animal models. In several embodiments, these methods include contacting a sample that includes isolated Plk1 with the test agent and contacting the sample with a peptide substrate of Plk1, such as the peptides disclosed herein, in presence of adenosine triphosphate, or an analog thereof, a for a period of time sufficient for Plk1 to phosphorylate the peptide in the absence of an inhibitor of Plk1 kinase activity. The presence of phosphorylated peptide is then detected to determine if the kinase activity of Plk1 is inhibited by the test agent. The presence of phosphorylated peptide would indicate that the test agent is not an inhibitor of Plk1 kinase activity. Alternatively, the presence of non-phosphorylated peptide can be detected to determine if the test agent is an inhibitor of Plk1 kinase activity. In some embodiments, the peptide is attached to a solid support. In some embodiments, the peptide is detectably labeled.

The amount of phosphorylated peptide and/or unphosphorylated peptide detected can be compared to a control. In several embodiments, the control is a known value indicative of the amount of phosphorylated peptide formed from basal phosphorylation of the peptide. In other embodiments, the control is a value indicative of the amount of phosphorylated peptide formed in the presence of a known amount of isolated Plk1. In still other embodiments, the control is the amount of phosphorylated peptide formed in a sample not contacted with the test agent. One of skill in the art will understand that the amount of unphosphorylated peptide can be determined from the difference between the total amount of peptide used and the amount of phosphorylated peptide detected.

A difference between the amount of phosphorylated peptide formed relative to a control can indicate that the test agent can be of use as an inhibitor of Plk1. For example, a test agent that decreases the amount of phosphorylated peptide formed relative to a control is identified as an inhibitor of Plk1 kinase activity. Similarly, a test agent that increases the amount of phosphorylated peptide formed relative to a control is identified as an activator (not an inhibitor) of Plk1 kinase activity.

In some embodiments, the difference between the amount of phosphorylated peptide in a sample contacted with a test agent relative to a control is a statistically significant difference. In some embodiments, the difference is at least about 10%, such as at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, at least about 500%, or greater then 500%. Thus, an agent can induce a statistically significant difference in the amount of complex formed in a sample contacted with a test agent relative to a control, such as a sample not contacted with the agent (such as an extract contracted with carrier alone).

### E. Exemplary Test Agents

The methods disclosed herein are of use for identifying agents that can be used to inhibit the kinase activity of Plk1. These agents are potential chemotherapeutics and could be used to treat cancer, for example a cancer in which Plk1 is expressed such as breast cancer, ovarian cancer, non-small cell lung cancer, head/neck cancer, colon cancer, endometrial cancer esophageal carcinomas, and leukemias.

An “agent” is any substance or any combination of substances that is useful for achieving an end or result. The agents identified using the methods disclosed herein can be of use for affecting the activity of Plk1, and can be of use for treating cancer. Any agent that has potential (whether or not ultimately realized) to affect the kinase activity of Plk1 can be tested using the methods of this disclosure.

Exemplary agents include, but are not limited to, peptides such as, soluble peptides, including but not limited to members of random peptide libraries (see, e.g., Lam et al., Nature, 354:82-84, 1991; Houghten et al., Nature, 354:84-86, 1991), and combinatorial chemistry-derived molecular library made of D- and/or L-configuration amino acids, phosphopeptides (including, but not limited to, members of random or partially degenerate, directed phosphopeptide libraries; see, e.g., Songyang et al., Cell, 72:767-778, 1993), antibodies (including, but not limited to, polyclonal, monoclonal, humanized, anti-idiotypic, chimeric or single chain antibodies, and Fab, F(ab′)₂ and Fab expression library fragments, and epitope-binding fragments thereof), small organic or inorganic molecules (such as, so-called natural products or members of chemical combinatorial libraries), molecular complexes (such as protein complexes), or nucleic acids.

Appropriate agents can be contained in libraries, for example, synthetic or natural compounds in a combinatorial library. Numerous libraries are commercially available or can be readily produced; means for random and directed synthesis of a wide variety of organic compounds and biomolecules, including expression of randomized oligonucleotides, such as antisense oligonucleotides and oligopeptides, also are known. Alternatively, libraries of natural compounds in the form of bacterial, fungal, plant and animal extracts are available or can be readily produced. Additionally, natural or synthetically produced libraries and compounds are readily modified through conventional chemical, physical and biochemical means, and may be used to produce combinatorial libraries. Such libraries are useful for the screening of a large number of different compounds.

Libraries (such as combinatorial chemical libraries) useful in the disclosed methods include, but are not limited to, peptide libraries (see, e.g., U.S. Pat. No. 5,010,175; Furka, Int. J. Pept. Prot. Res., 37:487-493, 1991; Houghton et al., Nature, 354:84-88, 1991; PCT Publication No. WO 91/19735), encoded peptides (e.g., PCT Publication WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Natl. Acad. Sci. USA, 90:6909-6913, 1993), vinylogous polypeptides (Hagihara et al., J. Am. Chem. Soc., 114:6568, 1992), nonpeptidal peptidomimetics with glucose scaffolding (Hirschmann et al., J. Am. Chem. Soc., 114:9217-9218, 1992), analogous organic syntheses of small compound libraries (Chen et al., J. Am. Chem. Soc., 116:2661, 1994), oligocarbamates (Cho et al., Science, 261:1303, 1003), and/or peptidyl phosphonates (Campbell et al., J. Org. Chem., 59:658, 1994), nucleic acid libraries (see Sambrook et al. Molecular Cloning, A Laboratory Manual, Cold Springs Harbor Press, N.Y., 1989; Ausubel et al., Current Protocols in Molecular Biology, Green Publishing Associates and Wiley Interscience, N.Y., 1989), peptide nucleic acid libraries (see, e.g., U.S. Pat. No. 5,539,083), antibody libraries (see, e.g., Vaughn et al., Nat. Biotechnol., 14:309-314, 1996; PCT App. No. PCT/US96/10287), carbohydrate libraries (see, e.g., Liang et al., Science, 274:1520-1522, 1996; U.S. Pat. No. 5,593,853), small organic molecule libraries (see, e.g., benzodiazepines, Baum, C&EN, Jan 18, page 33, 1993; isoprenoids, U.S. Pat. No. 5,569,588; thiazolidinones and methathiazones, U.S. Pat. No. 5,549,974; pyrrolidines, U.S. Pat. Nos. 5,525,735 and 5,519,134; morpholino compounds, U.S. Pat. No. 5,506,337; benzodiazepines, 5,288,514) and the like.

Libraries useful for the disclosed screening methods can be produce in a variety of manners including, but not limited to, spatially arrayed multipin peptide synthesis (Geysen, et al., Proc. Natl. Acad. Sci., 81(13):3998-4002, 1984), “tea bag” peptide synthesis (Houghten, Proc. Natl. Acad. Sci., 82(15):5131-5135, 1985), phage display (Scott and Smith, Science, 249:386-390, 1990), spot or disc synthesis (Dittrich et al., Bioorg. Med. Chem. Lett., 8(17):2351-2356, 1998), or split and mix solid phase synthesis on beads (Furka et al., Int. J. Pept. Protein Res., 37(6):487-493, 1991; Lam et al., Chem. Rev., 97(2):411-448, 1997). Libraries may include a varying number of compositions (members), such as up to about 100 members, such as up to about 1000 members, such as up to about 5000 members, such as up to about 10,000 members, such as up to about 100,000 members, such as up to about 500,000 members, or even more than 500,000 members.

In one convenient embodiment, high throughput screening methods involve providing a combinatorial chemical or peptide library containing a large number of potential therapeutic compounds. Such combinatorial libraries are then screened in one or more assays as described herein to identify those library members (particularly chemical species or subclasses) that display a desired characteristic activity (such as decreasing the measurable kinase activity of Plk1).

The compounds identified using the methods disclosed herein can serve as conventional “lead compounds” or can themselves be used as potential or actual therapeutics. In some instances, pools of candidate agents may be identified and further screened to determine which individual or subpools of agents in the collective have a desired activity.

### F. Kits and High Throughput Systems

This disclosure also provides kits for use in detecting the kinase activity of Plk1. The kits include one or more of the peptides disclosed herein. The kits may further include additional components such as instructional materials and additional reagents (for example isolate Plk1, specific binding agents, such as antibodies, for example antibodies that bind to Plk1, antibodies that specifically bind to the peptides disclosed herein, for example that specifically bind to the phospho-peptides or that specifically bind to the non-phospho peptides) radio nucleotides (such as ³²P-ATP) or the like). The kits may also include additional components to facilitate the particular application for which the kit is designed (for example microtiter plates). Thus, for example, the kit may additionally contain means of detecting a label (such as enzyme substrates for enzymatic labels, filter sets to detect fluorescent labels, appropriate secondary labels such as a secondary antibody, or the like). The instructional materials may be written, in an electronic form (such as a computer diskette or compact disk) or may be visual (such as video files).

This disclosure also provides integrated systems for high-throughput determination of Plk1 kinase activity, for example for high throughput screening of Plk1 inhibitors. The systems typically include a robotic armature that transfers fluid from a source to a destination, a controller that controls the robotic armature, a tag detector, a data storage unit that records tag detection, and an assay component such as a microtiter dish comprising a well having a reaction mixture.

A number of robotic fluid transfer systems are available, or can easily be made from existing components. For example, a Zymate XP (Zymark Corporation; Hopkinton, Mass.) automated robot using a Microlab 2200 (Hamilton; Reno, Nev.) pipetting station can be used to transfer parallel samples to 96 well microtiter plates to set up several parallel simultaneous assays.

Optional, optical images can viewed (and, if desired, recorded for future analysis) by a camera or other recording device (for example, a photodiode and data storage device) are optionally further processed in any of the embodiments herein, such as by digitalizing, storing, and analyzing the image on a computer. A variety of commercially available peripheral equipment and software is available for digitizing, storing and analyzing a digitized video or digitized optical image, e.g., using PC (Intelx86 or Pentium chip-compatible DOS™, OS2™ WINDOWS™, WINDOWS NT™ or WINDOWS95™ based computers), MACINTOSH™, or UNIX based (for example, a SUN™, a SGI™, or other work station) computers.

### G. Methods of Diagnosis

The poor prognosis subjects with cancers associated with Plk1 overexpression (and hence the hyperactivation of Plk1) could be improved if methods were available to identify such cancers in subjects prior to or early in their development, for example breast cancer, ovarian cancer, non-small cell lung cancer, head/neck cancer, colorectal cancer, hepatoblastoma, endometrial cancer, oropharyngeal carcinoma, esophageal carcinomas, melanoma, non-Hodgkin lymphoma and leukemia. This disclosure provides for diagnosing cancer associated with Plk1 expression including the predisposition for developing these cancers, for example prior to the onset of symptoms, and/or prior to the occurrence of morphological and physiological changes associated with malignancy.

Methods are provided herein for detecting a cancer, measuring the predisposition of a subject for developing a cancer, or determining the prognosis of the cancer. The methods include obtaining a biological sample from a subject and determining the Plk1 kinase activity present in the biological sample using the assays disclosed herein. In increase in the activity of Plk1 relative to a control indicates that the subject (from which the biological sample was obtained) has cancer is predisposed to developing cancer, or has a poor prognosis. A subject with increased Plk1 kinase activity relative to a control has for example breast cancer, ovarian cancer, non-small cell lung cancer, head/neck cancer, colon cancer, endometrial cancer, esophageal carcinomas, and leukemias is predisposed to developing these cancers, or has a poor prognosis. Thus, in some embodiments, the disclosed methods are used to detect a tumor in a subject, for example by detecting an increase in Plk1 kinase activity relative to the basal level of Plk1 kinase activity in a biological sample obtained from a subject.

The subject can be any subject of interest, including a human subject. In some embodiments, a subject of interest is selected, for example a subject who has cancer, for example to determine the prognosis of such a subject, or a subject in need of a diagnosis of cancer.

In some examples, the sample that is tested for Plk1 kinase activity is a biological sample. In some examples, a biological sample is obtained from an organism or a part thereof, such as an animal. In particular embodiments, the biological sample is obtained from an animal subject, such as a human subject. A biological sample can be any solid or fluid sample obtained from, excreted by or secreted by any living organism, including without limitation multicellular organisms, such as mammals. In some embodiments, a biological sample is obtained from a human subject, such as an apparently healthy human subject or a human patient affected by a condition or disease to be diagnosed or investigated, such as cancer. In some examples, a biological sample obtained from a subject a sample obtained from an organ or tissue of the subject, for example a biopsy specimen, such as a tumor biopsy. In some examples, a biological sample is a cell lysate, for example a cell lysate obtained from the tumor of a subject.

The methods disclosed herein are particularly suited for monitoring disease progression in a subject. Thus, disclosed are methods for determining the prognosis of a subject suffering from cancer. In some embodiments, such methods involve detecting the kinase activity of Plk1 at a first time point, and detecting the kinase activity of Plk1 at a second time point, and comparing the kinase activity of Plk1 at the two time points. If a decrease in the kinase activity of Plk1 at the second time point is detected, the subject is showing signs of disease remission. Conversely, if an increase in the kinase activity of Plk1 is observed at the second time point the subject is showing signs of disease progression. In some embodiments, the disclosed methods are used to predict the prognosis of a subject suffering from cancer, for example by measuring the kinase activity of Plk1 at a first time point and a second time point. If a decrease in the kinase activity of Plk1 at the second time point is detected, the subject is showing signs of disease regression. Conversely, if an increase in the kinase activity of Plk1 is observed at the second time point the subject is showing signs of disease progression. In particular examples, a cancer in which Plk1 is expressed is breast cancer, ovarian cancer, non-small cell lung cancer, head/neck cancer, colon cancer, endometrial cancer esophageal carcinomas, and leukemias.

The methods disclosed herein can be used to monitoring efficacy of treatment of disease, for example the treatment of a cancer in which Plk1 is expressed is breast cancer, ovarian cancer, non-small cell lung cancer, head/neck cancer, colon cancer, endometrial cancer esophageal carcinomas, and leukemias. In some embodiments, such methods involve detecting the kinase activity of Plk1 at a first time point (for example prior to treatment or after treatment has begun) and detecting the kinase activity of Plk1 at a second later time point (for example later in the treatment cycle or after treatment has ended) and comparing the kinase activity of Plk1 at the two time points. If a decrease in the kinase activity of Plk1 at the second time point is detected, the subject is showing signs of responding to the treatment. Conversely, if an increase in the kinase activity of Plk1 is observed at the second time point this indicates that the treatment may not be effective and it may be advantageous for to select a different treatment. Also encompassed by this disclosure are methods for selecting a treatment regimen or therapy for the prevention, reduction, or inhibition of cancer. In some examples, these methods involve detecting an increase in the kinase activity of Plk1 in a subject, and if such decrease is detected, a treatment is selected to prevent or reduce cancer or to delay the onset cancer. The subject then can be treated in accordance with this selection. Such treatments include without limitation the use of chemotherapeutic agents, immunotherapeutic agents, radiotherapy, surgical intervention, or combinations thereof.

The following examples are provided to illustrate certain particular features and/or embodiments. These examples should not be construed to limit the invention to the particular features or embodiments described.

### EXAMPLES

### Example 1

### Construction of GST-PBIPtides

This example describes exemplary techniques to construct PBIPtides used in subsequent studies.

To generate GST-PBIPtide expressing constructs, a pUC19 derivative, pUC19N, was generated in which multiple cloning sites were restructured to contain both BamHI and BglII. A small DNA fragment encoding GGPGG (SEQ ID NO: 12) fused-YETFDPPLHSTAIYADEE (amino acid residues 68-85 of SEQ ID NO: 10) (PBIPtide) was digested with BamHI (5′ end) and BglII (3′ end) and then inserted into pUC19N digested with the corresponding enzymes. The GGPGG (SEQ ID NO: 12) sequence was added at the N terminus of the PBIPtide as a linker between PBIPtide repeats. The resulting pUC19NGGPGG-PBIPtide was digested with BglII and then ligated with another copy of GGPGG-PBIPtide digested with BamHI (5′ end) and BglII (3′ end), yielding pUC19N-GGPGG-PBIPtide2. These cloning steps were repeated 2 more times to generate pUC19N-GGPGG-PBIPtide4 (this cloning strategy disables the N-terminal BamHI site of the subsequent GGPGG-PBIPtide fragments inserted). The last GGPGG-PBIPtide fragment bears a stop codon to terminate translation. To generate GST-GGPGG-PBIPtide4 construct (for simplicity, this construct id referred to as GST-PBIPtide4), pUC19N-GGPGG-PBIPtide4 was digested with BamH1 and BglII and then inserted into pGEX-4T-2 (Amersham Biosciences) digested with BamH1.

To eliminate potential phosphorylation sites other than the T78 residue, PBIPtide-A form (FEAFDPPLHSTAIFADEE, SEQ ID NO: 4) that bears 3 mutations (Y68F, T70A, and Y81F) was generated. Construction of GST-PBIPtide-A6, which contains 6 copies of the GGPGG-PBIPtide-A fragment, was carried out in a similar manner as described. GST-PBIPtides were expressed and purified from E. coli BL21 by using glutathione (GSH)-agarose (Sigma).

### Example 2

### Exemplary Plk1 Kinase Assay Using GST-PBIPtide as a Plk1 Affinity Ligand and in vitro Substrate

This example describes tests for Plk1 kinase activity using a GST-PBIPtide as a Plk1 affinity ligand and in vitro substrate of Plk1.

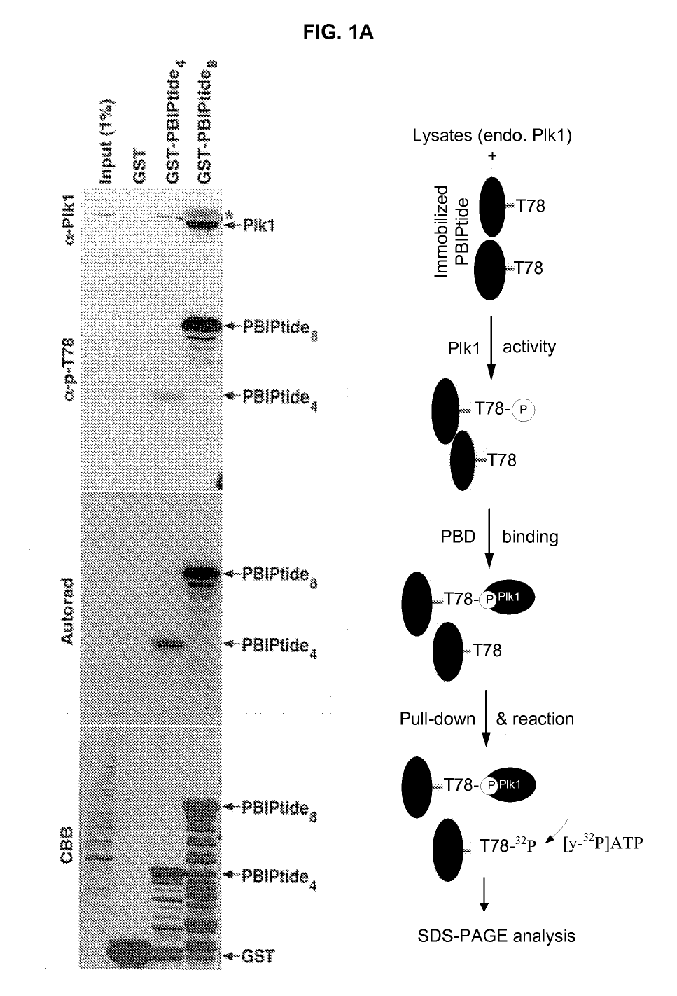

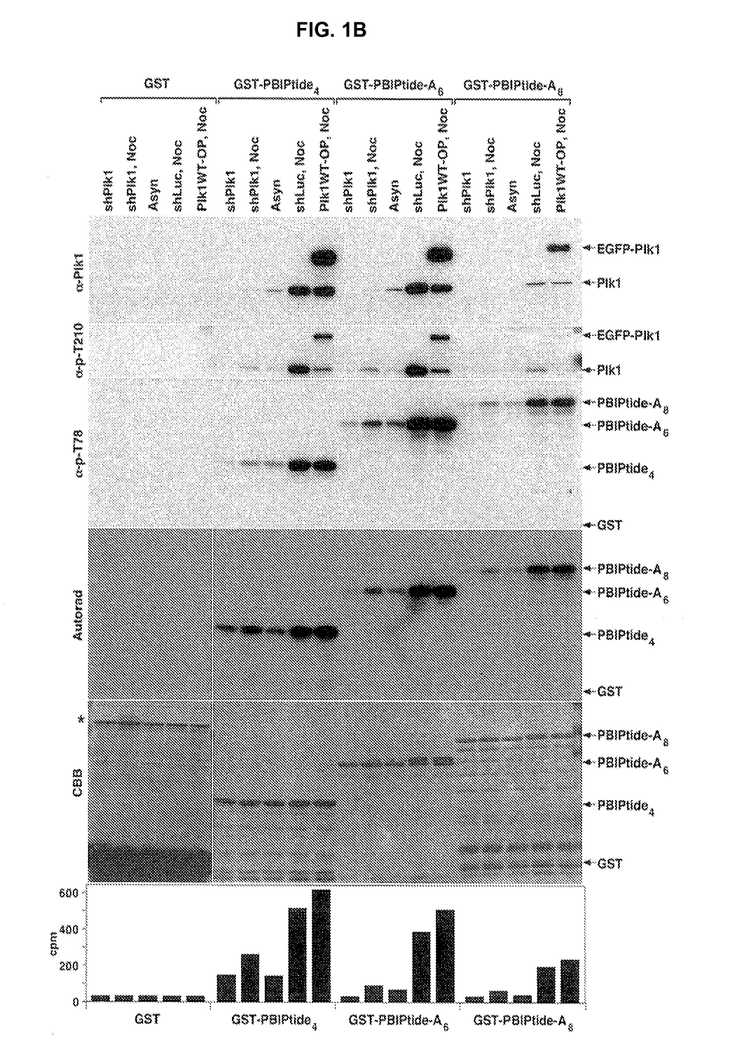

Plk1 efficiently phosphorylates a centromeric protein PBIP1 at T78, and this phosphorylation event generates a docking site for a high-affinity interaction between the PBD of Plk1 and p-T78 PBIP1 (Kang et al. (2006) Mol Cell 24:409-422). Subsequent investigation revealed that none of the other mitotic kinases tested (Cdc2, Aurora A, Aurora B, Mps1, and Erk1) appeared to phosphorylate the T78 residue of PBIP1. By taking advantage of the specific Plk1-dependent PBIP1 phosphorylation and subsequent interaction between the resulting p-T78 epitope and the PBD, it was examined whether a GST-fused PBIP1 peptide bearing the T78 motif (hereon referred to as GST-PBIPtide) could precipitate Plk1 through the Plk1-generated p-T78 epitope and whether the precipitated Plk1 could further phosphorylate as yet unphosphorylated GST-PBIPtide. To develop this assay a GSTPBIPtide4 (PBIPtide was first generated containing 4 repeats of the T78 motif to enhance the phosphorylation level and binding affinity) and expressed it in Escherichia coli (see Example 1). The resulting bead-associated GSTPBIPtide4 was incubated with mitotic HeLa lysates in a conventional immunoprecipitation buffer, precipitated, and then reacted in a kinase reaction mixture (KC buffer) in the presence of [γ-³²P]ATP. The GST-PBIPtide coprecipitated Plk1, which, in turn, phosphorylated and generated the p-T78 epitope on neighboring GST-PBIPtide molecules (FIG. 1A). Under the same conditions, the control GST failed to precipitate Plk1 and generate the p-T78 epitope (FIG. 1A). As expected, if the Plk1 activity in the total lysates was responsible for generating the p-T78 epitope, Plk1 immunoprecipitated from cultured lysates efficiently phosphorylated GST-PBIPtide and generated the p-T78 epitope in a kinase activity-dependent manner.

As shown in FIG. 1A, a GST-PBIPtide was able to act as an in vitro Plk1 substrate and precipitate Plk1. Mitotic HeLa lysates were incubated in TBSN buffer containing phosphatase inhibitors with either bead-immobilized control glutathione S-transferase (GST) or the GST-PBIPtides as indicated in FIG. 1A. Beads were precipitated, washed, and then subjected to in vitro kinase assays in the presence of [γ-³²P]-ATP. Samples were separated by gel electrophoresis using 10% sodium dodecyl sulfate polyacrylamide gel electrophoresis (SDS-PAGE) gels, transferred to polyvinylidene fluoride (PVDF) transfer membranes, and then the amount of incorporated ³²P was determined by autoradiography. Subsequently, the same membrane was immunoblotted with anti-Plk1 or anti-p-T78 antibodies, and then stained with Coomassie stain. The peptides used in this study are denoted as GST-PBIPtide₄ (N-terminal GST fusion of SEQ ID NO: 4) and GST-PBIPtide₈, (N-terminal GST fusion of SEQ ID NO: 10) where the number refers to the number of repeats of the T78 motif present in the GST-fused PBIPtide.

It was then determined whether the Plk1 activity present in total cellular lysates could be directly measured by incubating the lysates with GST-PBIPtide. Total cellular lysates were prepared from either control luciferase RNAi (shLuc) or Plk1 RNAi (shPlk1)-treated HeLa cells in a KC-plus buffer (kinase reaction mixture supplemented with 100 mM NaCl, 0.5% Nonidet P-40, and protease inhibitors), mixed with GST-PBIPtide4, and then reacted in the presence of 15 μCi of [γ-³²P]ATP. Consistent with the levels of Plk1 expression (FIG. 1B, α-Plk1) and activational Plk1 phosphorylation at T210 (FIG. 1B, α-p-T210), the control shLuc cells arrested with nocodazole (M phase) exhibited a high level of T78 phosphorylation onto PBIPtide4 (FIG. 1B; α-p-T78 in the center column). By contrast, asynchronously growing shLuc cells (Asyn) displayed a significantly diminished level of the p-T78 epitope. Cells silenced for Plk1 (shPlk1) showed a very low level of T78 phosphorylation. However, the same sample exhibited a much higher level of ³²P incorporation onto PBIPtide4 (FIG. 1B; compare the autorad with the α-p-T78 blot in the center column), suggesting that the Thr and Tyr residues other than the T78 residue within PBIPtide4 are phosphorylated during the incubation with total lysates. To eliminate these potential phosphorylation sites, the Y68, T70, and Y81 sites were mutated within the GST-PBIPtide (thus leaving the T78 residue as the only phosphorylation site) to generated a GST-PBIPtide variant bearing 6 repeats of the PBIPtide (Y68F T70A Y81F) triple mutant (herein referred to as GST-PBIPtide-A6). Examination of Plk1-dependent GST PBIPtide-A6 phosphorylation showed that the level of the p-T210 epitope (a measure of Plk1 activity) was closely correlated with that of the p-T78 epitope on PBIPtide-A6 (FIG. 1B; compare the 2nd and 3rd panels in the right column). Additionally, PBIPtide-A6 displayed a mildly, but reproducibly, lower level of ³²P incorporation after Plk1 depletion than the original PBIPtide4 (FIG. 1B; autorad in the right column), indicating an improved sensitivity to Plk1 activity.

The anti-Plk1 and anti-p-T78 immunoblots indicate the total amount of Plk1 co-precipitated with the GST-PBIPtide and the level of the p-T78 epitope (present in the phosphorylated PBIPtide) among the Plk1 precipitates, respectively. In this study GST-PBIPtide₄ (PBIPtide with four repeats of the T78 motif), GST-PBIPtide-A₆ (N-terminal GST fusion of SEQ ID NO: 8) and GST-PBIPtide-A₈ (PBIPtides with six or eight repeats, respectively, of a T78 motif containing mutations corresponding to Y68F, T70A, and Y81F were used (PBIP1 numbering)) were used.

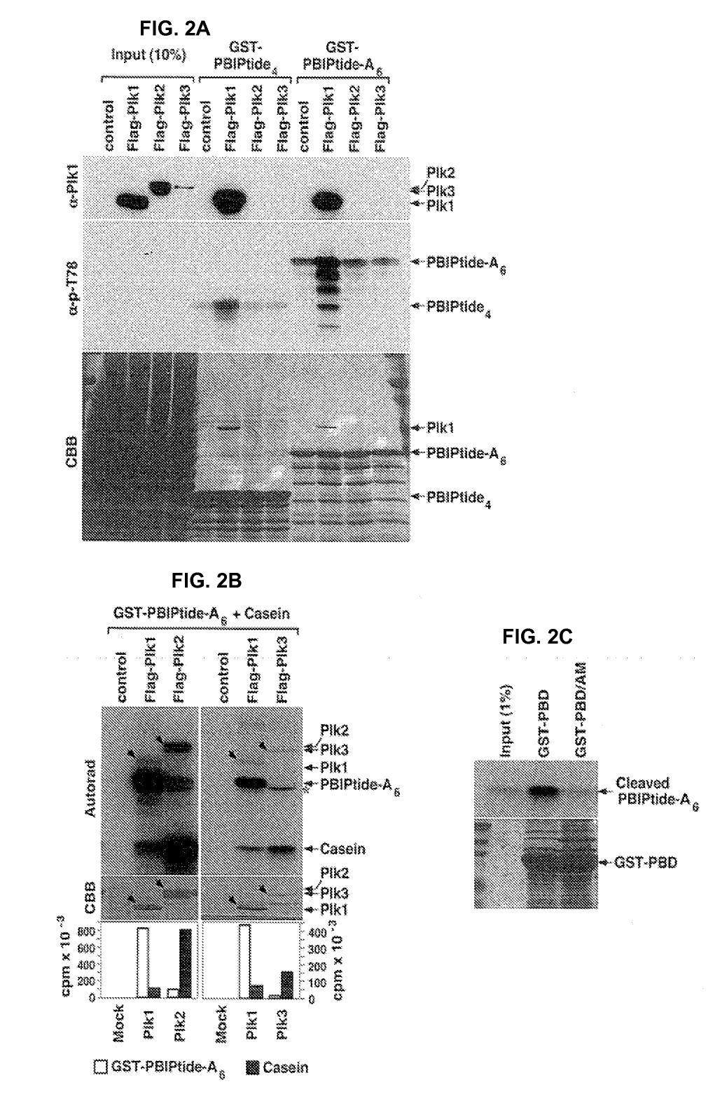

For GST-PBIPtide pulldown kinase assays, HeLa cells were lysed in TBSN buffer [20 mMTris-Cl (pH8.0), 150 mMNaCl, 0.5% Nonidet P-40, 5 mM EGTA, 1.5 mM EDTA, 20 mM p-nitrophenyl phosphate, and protease inhibitor mixture (Roche]. The resulting lysates were clarified by centrifugation at 15,000×g for 20 minutes at 4° C. and then incubated with bead-bound GST-PBIPtide to precipitate p-T78 PBIPtide-bound Plk1. For efficient Plk1 precipitation with bead-bound GST-PBIPtide in FIG. 2A, total cellular lysates were prepared in a KC-plus [kinase reaction mixture (KC) supplemented with 100 mM NaCl, 0.5% Nonidet P-40, and protease inhibitors] buffer that allows more efficient substrate phosphorylation than the TBSN buffer. For ELISAs, GST-PBIPtide bound to the agarose resin was eluted with glutathione (GSH), and then dialyzed before use.

To directly measure the Plk1 kinase activity with total cellular lysates, HeLa cells were lysed in KC-plus buffer and then clarified by centrifugation at 15,000×g for 20 minutes at 4° C. The resulting cellular proteins (˜200 μg) were incubated with bead-bound control GST or GST-PBIPtide in the presence of 10 μCi of [γ-³²P]ATP (1 Ci=37 GBq) at 30° C. for 30 minutes. Reactions were terminated by the addition of a large volume of cold KC-plus buffer. Beads were washed with KC-plus buffer and then mixed with SDS/PAGE sample buffer. The resulting samples were separated by 10% SDS/PAGE, transferred to polyvinylidene fluoride (PVDF) (Millipore), and exposed (Autorad). After immunoblotting, the same membranes were stained with Coomassie (CBB). Protein bands were excised and incorporated ³²P was measured by liquid scintillation spectrometry.

To examine the interaction between PBD and the p-T78 of PBIPtide-A6, bead-bound GST-PBIPtide was first reacted with immunoprecipitated Plk1, Plk2 or Plk3 with constant agitation in the presence of [γ-³²P]ATP. Subsequently, the reacted GST-PBIPtide was digested with thrombin (INVITROGEN®), and then the resulting soluble ³²P-labeled PBIPtide-A6 was incubated with the bead-bound, bacterial, GST-PBD or GST-PBD (H538A K540M) in TBSN for 1 hour at 4° C. Precipitates were washed in the binding buffer, separated by SDS/PAGE, stained with Coomassie (CBB), and then exposed (Autorad).

Primary antibodies used for this study were anti-PBIP1 p-T78 antibody (Rockland Immunologicals, Inc.), anti-Plk1 antibody (F-8) (Santa Cruz Biotechnology), anti-Plk1 p-T210 antibody (BD Biosciences), anti-Flag antibody (Sigma), anti-GFP antibody (MBL International), anti-Plx1 antibody, and anti-Cyclin B1 antibody (Abcam, Inc.). Proteins that interact with the primary antibodies were decorated with appropriate HRP-conjugated secondary antibodies and detected by using the enhanced chemiluminescence (ECL) western detection system.

Cell cultures were maintained as recommended by American Type Culture Collection. Plasmid transfection was carried out by using LIPOFECTAMINE® 2000 (INVITROGEN®). Lentiviruses expressing shRNA were generated as described in Kang et al. (Kang et al. (2006) Mol Cell 24:409-422). The target sequences for shPlk1 and shPlk3 were reported previously (Hansen et al. (2004) Mol Biol Cell 15:5623-5634; Zimmerman and Erikson (2007) Proc Natl Acad Sci USA 104:1847-1852). HeLa cells were infected with the indicated lentiviruses for 1 day and further incubated with fresh medium for an additional 2 days before harvest. Where indicated, cells were treated with 2.5 mM thymidine (Sigma) or 200 ng/ml of nocodazole (Sigma) for 16 hours to arrest the cells in S or M phase, respectively. To acutely inhibit the Plk1 kinase activity, HeLa cells were treated with 100 nM of BI 2536 for 30 minutes before harvest.

### Example 3

### Specificity of PBIPtides for Plk1

This example describes tests for the specificity of the GST-PBIPtides disclosed herein for Plk1.

As shown in FIG. 2A-2C, Plk1, but not Plk2 or Plk3, phosphorylates and binds to the T78 motif of GST-PBIPtides. HeLa cell lysates obtained from HeLa cells expressing either Flag-Plk1, Flag-Plk2, or Flag-Plk3 were prepared in KC-plus buffer, and then incubated with the GST-PBIPtides (indicated in FIG. 2A-2C) immobilized on glutathione (GSH) agarose beads. The GST-PBIPtide precipitates were separated by SDS-PAGE, transferred to a PVDF membrane, and then immunoblotted with anti-Plk1 and anti-phospho-T78 antibody (see FIG. 2A). Afterward, the same membrane was stained with Coomassie (CBB) (see FIG. 2A). Note that the lysates expressing Plk1 efficiently generated the phospho-T78 epitope on GST-PBIPtides and, as a result, bound Plk1. The low levels of the phospho-T78 signals from the Plk2 or Plk3 transfected cells are likely due to the endogenous Plk1 present in these samples. As shown in FIGS. 2B and 2C, Plk1-dependent phosphorylation of the T78 motif present in the PBIPtides was sufficient to bind to the phospho-peptide binding cleft of the PBD. Flag-Plk1, Flag-Plk1, and Flag-Plk3 immunoprecipitates were prepared from the respective transfected 293T cells (i.e. 293T cells expressing the Flag tagged Plks).

The immunoprecipitates were subjected to in vitro kinase assays using both GST-PBIPtide and casein as substrates in the same reaction tube. Samples were separated by 10% SDS-PAGE for autoradiography (see FIG. 2B). The arrows in FIG. 2B indicate autophosphorylated signals corresponding to Plk1, Plk2, or Plk3 in the gels stained with Commassie (CBB). The bands were excised and incorporated ³²P was measured by liquid scintillation counter (see bottom of FIG. 2B). The Plk1-phosphorylated, bead-bound, GST-PBIPtide was digested with thrombin. The resulting soluble ³²P-PBIPtide was incubated with either bead-bound GST-PBD or GST-PBD (H538A K540M) in TBSN buffer (20 mM Tris (pH 8.0° mM NaCl/0.5% Nonidet P-40/5 mM EGTA/1.5 mM Et 0.5 mM Na₃V0₄/20 mM p-nitrophenyl phosphate) for 1 hour at 4° C. Precipitates were washed extensively with the binding buffer and then analyzed as in as above (see FIG. 2C). These results demonstrate that the disclosed PBIPtides are highly specific for Plk1 at the exclusion of other kinases, including the related kinases Plk2 and Plk3.

### Example 4

### Development Plk1 Specific ELISA Assay

This example describes the development of an exemplary ELISA assay to measure the kinase activity of Plk1.

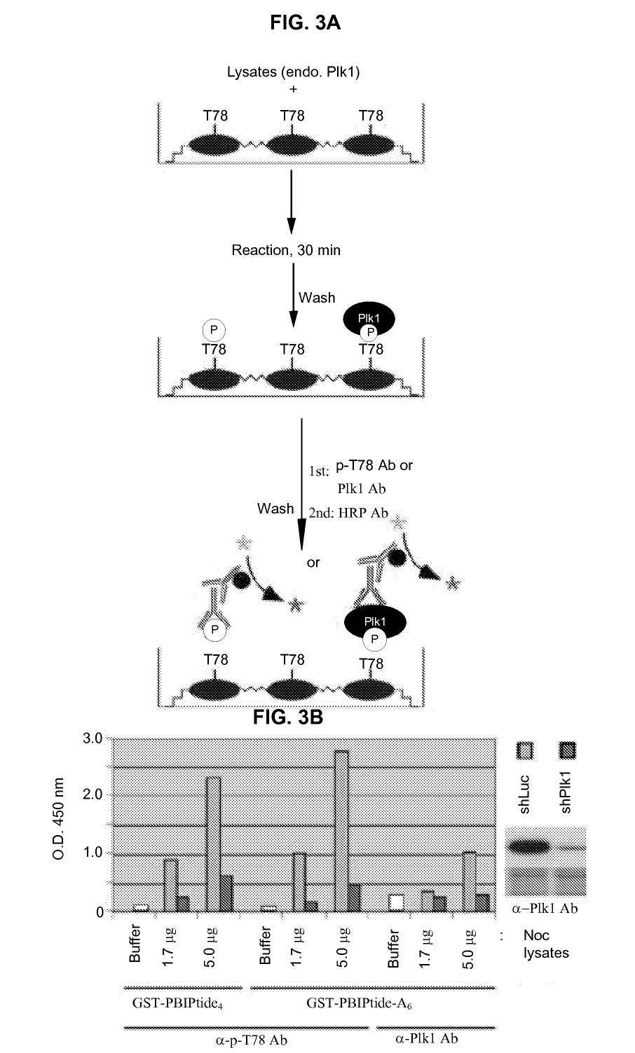

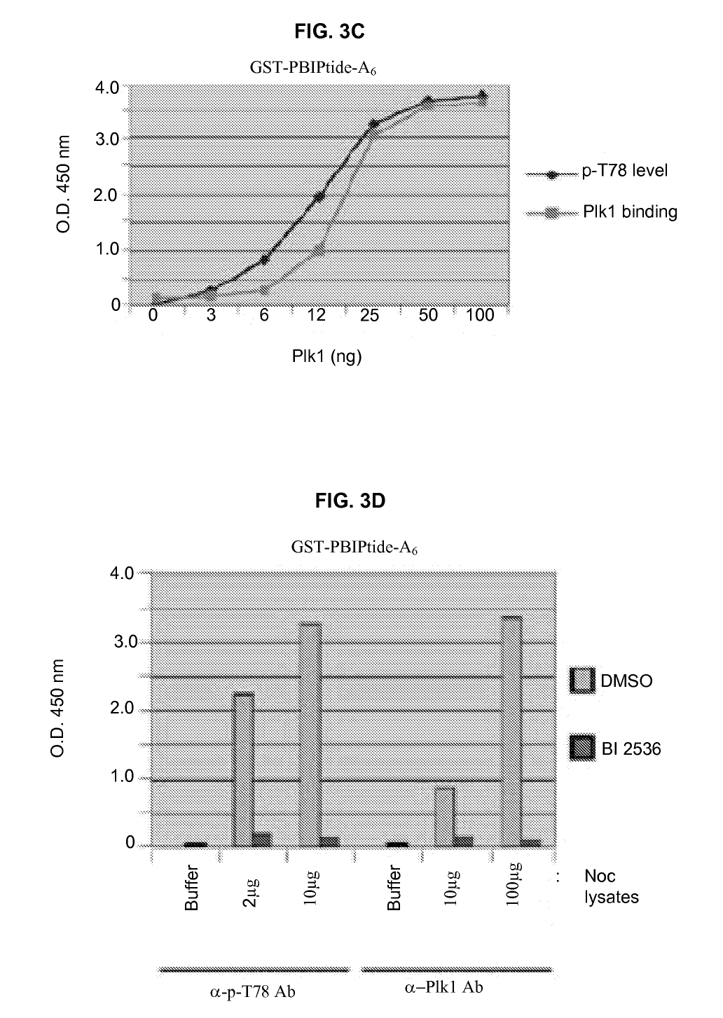

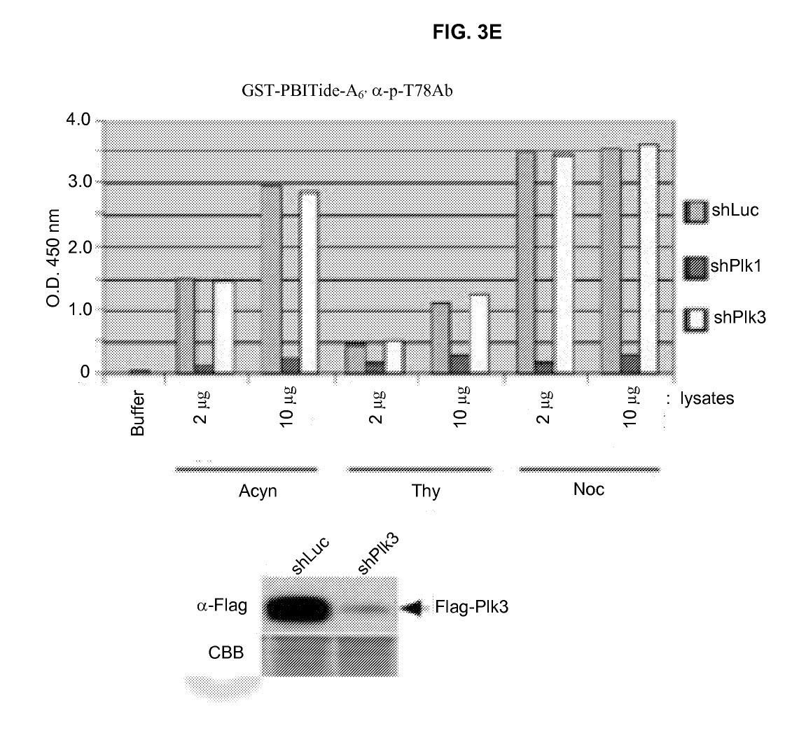

By exploiting the high specificity in Plk1-dependent PBIPtide phosphorylation and the ensuing PBD-p-T78 interaction, a PBIPtide-based ELISA was designed to measure the Plk1 activity by either of 2 approaches: (i) quantifying the level of the p-T78 epitope (α-p-T78 antibody) or (ii) measuring the level of Plk1 binding (α-Plk1 antibody) (FIG. 3A). Shown in FIG. 3A is a schematic illustrating an exemplary ELISA assay for measuring Plk1 kinase activity. With reference to FIG. 3A, the Plk1 activity present in the total cellular lysates generates the phospho-T78 epitope to which Plk1 itself binds. Either an anti-phospho-T78 antibody (to detect the phospho-T78 epitope generated) or an anti-Plk1 antibody (to detect Plk1 bound to the phospho-T78 epitope) can be used to detect the phospho-peptide. As shown in FIG. 3B, Plk1 generates the phospho-T78 and binds to the phospho-T78 epitope in a concentration-dependent manner.

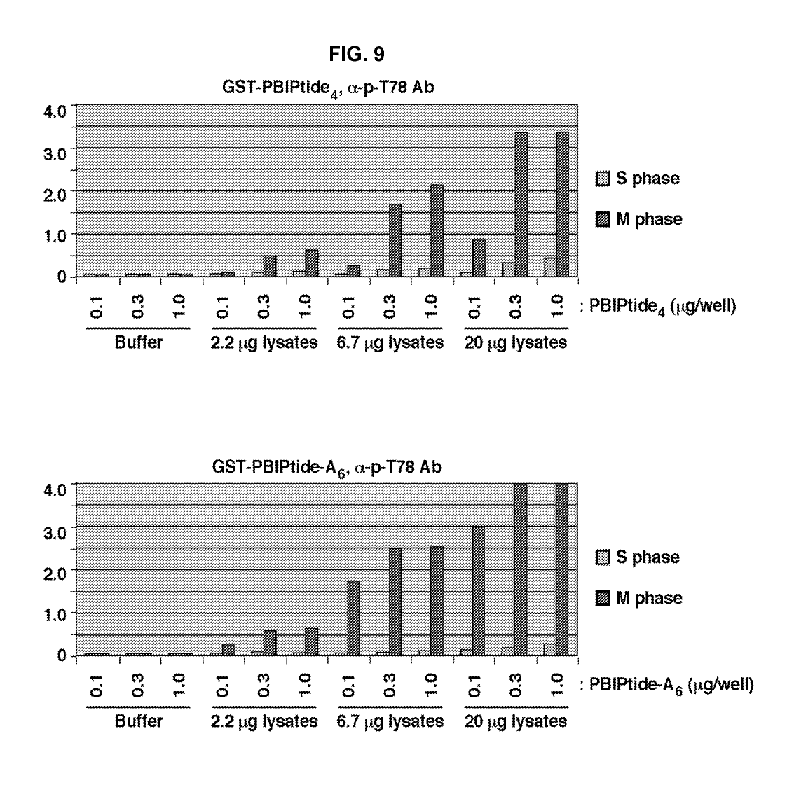

To perform these assays, a 96-well plate was coated with various amounts of GST-PBIPtide containing the T78 motif and then applied with a range of total cellular lysates prepared in the KC-plus buffer to allow efficient phosphorylation onto PBIPtide during incubation (˜2-10 μg of total HeLa lysates were sufficient for reactions with ˜0.5-1 μg of GST-PBIPtide4 or GST-PBIPtide-A6 per well; see FIGS. 9A and 9B). Attesting to the specificity of Plk1-dependent PBIPtide phosphorylation, depletion of Plk1 greatly diminished the levels of both the p-T78 epitope and bound Plk1 on GST-PBIPtides (FIG. 3B). Consistent with the results shown in FIG. 1B, PBIPtide-A6 exhibited a mildly, but reproducibly, increased sensitivity to Plk1 activity compared with PBIPtide4. Notably, the level of the Plk1 binding (α-Plk1 signal) was significantly lower than the level of the p-T78 generation (α-p-T78 signal) (FIG. 3B), in part because Plk1 binds to a fraction of the total p-T78 epitope generated. Because of the apparently elevated sensitivity, GST-PBIPtide-A6 was chosen for further analysis. To verify the Plk1-dependent PBIPtide phosphorylation and subsequent PBD binding, the above assay was repeated using purified recombinant proteins. Results showed that the level of the p-T78 epitope generated was proportional to the amount of Plk1 provided. Although the Plk1 amounts that can be quantified in a linear range could be relatively narrow because of the nature of an ELISA-based assay, the increase in the level of the p-T78 epitope also closely paralleled the increase in the amount of Plk1 bound to PBIPtide (FIG. 3C). In a second test, it was also found that treatment of cells with a Plk1 inhibitor, BI 2536, for 30 min was sufficient to acutely inhibit the Plk1-dependent p-T78 generation and Plk1 binding (FIG. 3D), further confirming that Plk1 activity is responsible for these events.

It has been shown that Plk2 is transiently expressed during the G0/G1 transition. However, Plk3 is reported to be active in S and G2 phases of the cell cycle, during which Plk1 is also progressively accumulated and activated before mitotic entry. Furthermore, overexpression of Plk3 rescues the growth defect associated with the budding yeast polo kinase cdc5-1 mutation. Thus, it was examined whether Plk3 contributes to the generation of the p-T78 epitope in vivo. Consistent with Plk1-dependent PBIPtide phosphorylation, the level of p-T78 was high in nocodazole-treated (M phase) cells but low in thymidine-arrested (S phase) cells (FIG. 3E). Depletion of Plk1 drastically diminished the level of the p-T78 epitope, whereas depletion of Plk3 did not significantly alter the level of the p-T78 epitope under various conditions examined (FIG. 3E). These results suggest that Plk3 does not contribute to the generation of the p-T78 epitope.

To coat a 96-well plate (Beckman-Coulter) with PBIPtide, soluble GST-PBIPtide4 or GST-PBIPtide-A6 was first diluted with 1× coating solution (KPL Inc.) to an optimal concentration (10 μg/ml). The resulting solution was then added into each well (50 μL per well) and incubated for 12-18 hours at room temperature. To block the unoccupied sites, wells were washed once with PBS plus 0.05% Tween20 (PBST), and then incubated with 200 μL of PBS plus 1% BSA for 1 hour. The PBIPtide phosphorylation reaction was carried out with 100 μL of total cellular lysates in KC-plus buffer or the indicated amount of recombinant Flag-Plk1 from Sf9 cells for 30 minutes at 30° C. on an ELISA plate incubator (Boekel Scientific). To terminate the reaction, ELISA plates were washed 4 times with PBST. For detection of the generated p-T78 epitope or bound Plk1, plates were incubated for 2 hours with 100 μL per well of anti-p-T78 or anti-Plk1 antibody at a concentration of 0.5 μg/ml. After washing the plates 5 times, 100 μL per well of HRP-conjugated secondary antibody (diluted 1:1,000 in blocking buffer) was added and the plates incubated for 1 hour. Plates were then washed 5 times with PBST and then incubated with 100 μL per well of 3,3′,5,5′-tetramethylbenzidine solution (TMB) (Sigma) as substrate until a desired absorbance was reached. The reactions were stopped by the addition of 0.5M H₂SO₄. The optical density of the samples was measured at 450 nm by using an ELISA plate reader (Molecular Devices).

### Example 5

### Comparison of Conventional Immunocomplex Kinase Assay and the Disclosed ELISA Assay

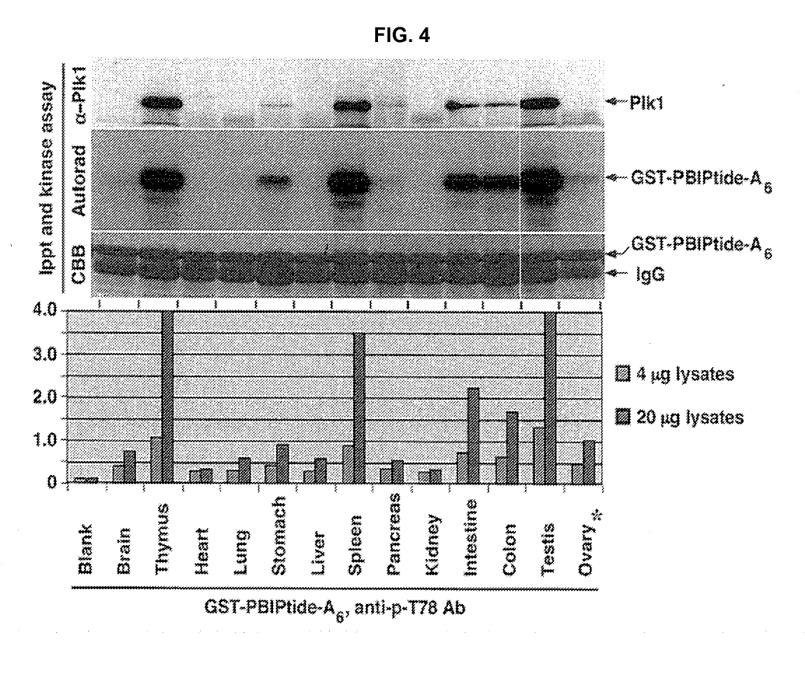

The example describes that the kinase activity of Plk1 as measured using the ELISA assay disclosed herein is far more sensitive than a conventional immunocomplex kinase assay. Total cellular proteins were prepared from the indicated tissues obtained from 1.5 month-old male and its littermate sister (for ovary only) mice. Anti-Plk1 immunoprecipitation kinase assays were carried out with 2 mg of total lysates for each tissue (except ovary), whereas Plk1 ELISA assays were performed with either 4 μg or 20 μg of the same lysates. For in vitro kinase assays, reacted samples were separated by SDS-PAGE and transferred to a PVDF membrane for autoradiography and immunoblot analysis (see FIG. 4). The same membrane was then stained with Coomassie (CBB). The asterisk in FIG. 4 indicates that only half amounts of total lysates (1 mg) and anti-Plk1 antibody (3 μg) were used for immunoprecipitation due to the limited amount of the ovary tissues. ELISA assays were carried out as described using GST-PBIPtide-A₆ as Plk1 substrate. These results demonstrate that the disclosed ELISA is approximately 100 times more sensitive than a conventional immunocomplex kinase assay.

### Example 6

### Direct Measurement of Plk1 Activity in Mouse Tissues

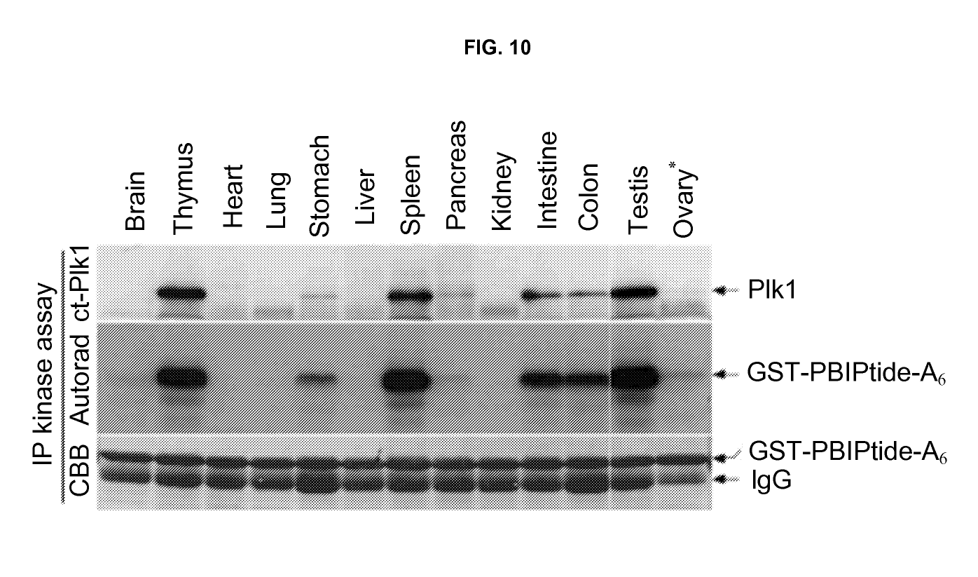

To determine whether the above PBIPtide-based ELISA could be used to measure the level of Plk1 activity in mammalian tissues, a comparison was made between the ELISA and the conventional immunocomplex kinase assay using the same lysates prepared from various mouse tissues. It was found that the Plk1 activities from the ELISA showed a tight correlation with those from the immunocomplex kinase assays (FIG. 4A; see also immunocomplex kinase assays using GST-PBIPtide-A6 as substrates in FIG. 10). Remarkably, only 4-20 μg of the same lysates (because of low mitotic indices for tissues, more lysates were needed from tissues than from cultured cells) were used for the ELISA compared with 2 mg of total lysates for the immunoprecipitation kinase assay. Thus, the Plk1 ELISA is not only rapid but also sensitive, allowing accurate quantification of Plk1 activity with 100- to 500-fold smaller amounts of total lysates from cells and tissues.

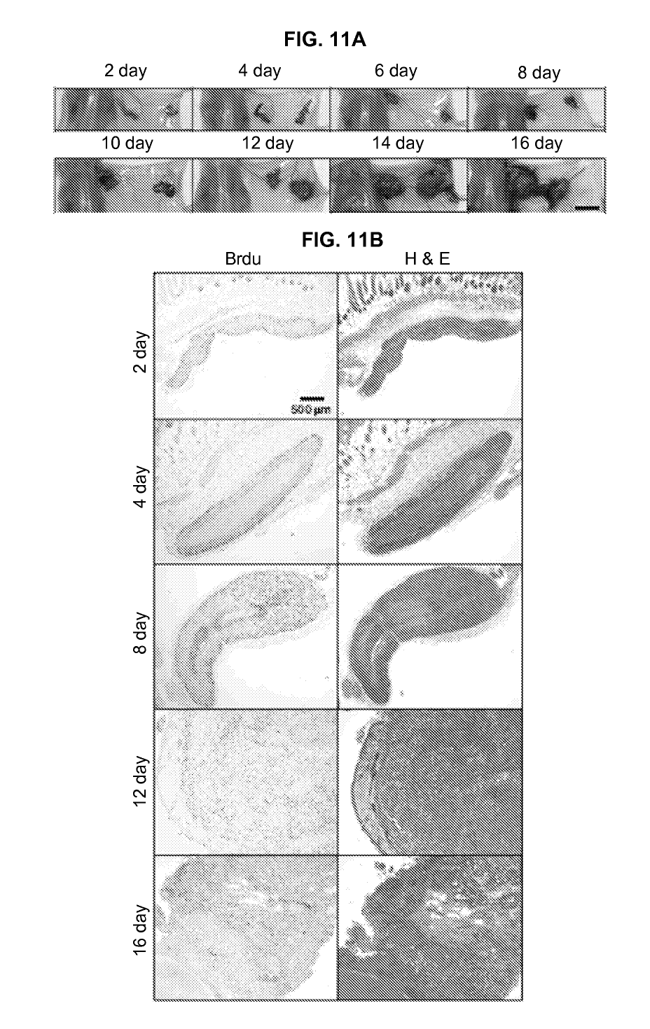

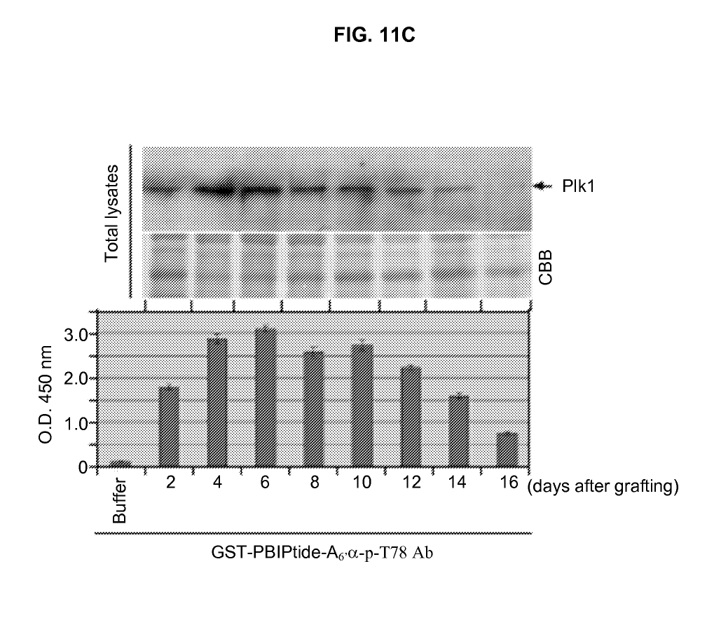

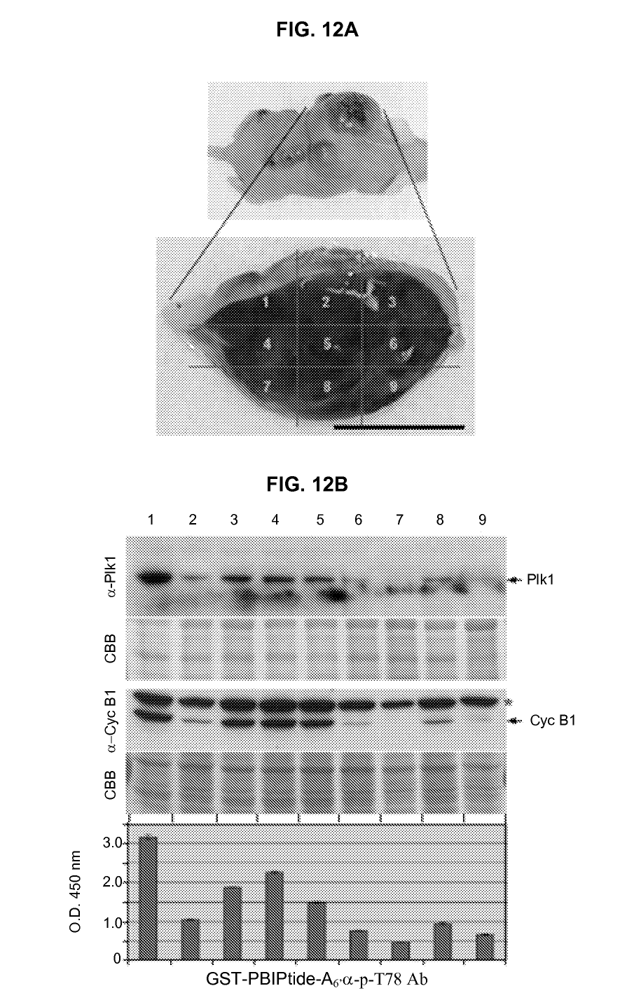

Taking advantage of this highly sensitive assay, it was then investigated whether Plk1 activity alters during tumorigenesis using B16-derived xenografted tumors in nude mice (FIG. 4B). Plk1 activity was low immediately after grafting but soon reached a maximum level during early stages of tumorigenesis (FIG. 4C). However, the level of Plk1 activity began to diminish as the tumor reached a significant volume (˜10 mmin diameter). The levels of Plk1 activity closely mirrored those of Plk1 expression (FIG. 4C) and cell proliferation activity (FIG. 11). Measurement of Plk1 activity of tissues taken from different parts of a single tumor revealed that the levels of Plk1 expression and activity tightly correlated with the level of mitotic Cyclin B1 (FIG. 12). These findings suggest that elevated Plk1 activity is critical during early stages of tumorigenesis and strongly support the view that the level of Plk1 functions as an indicator of cell proliferation.

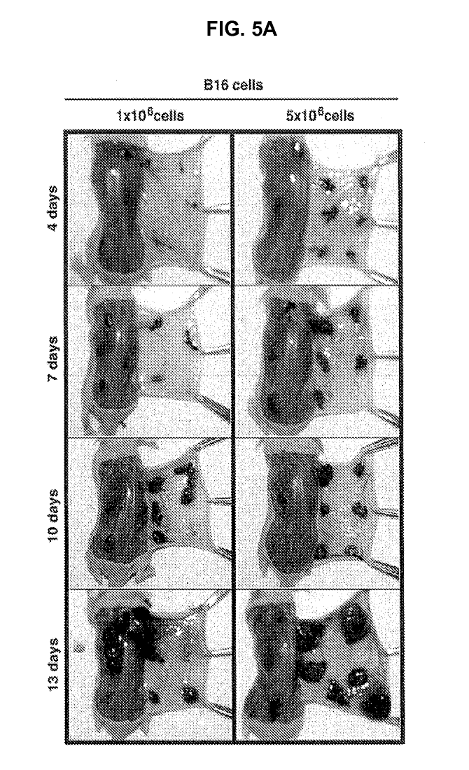

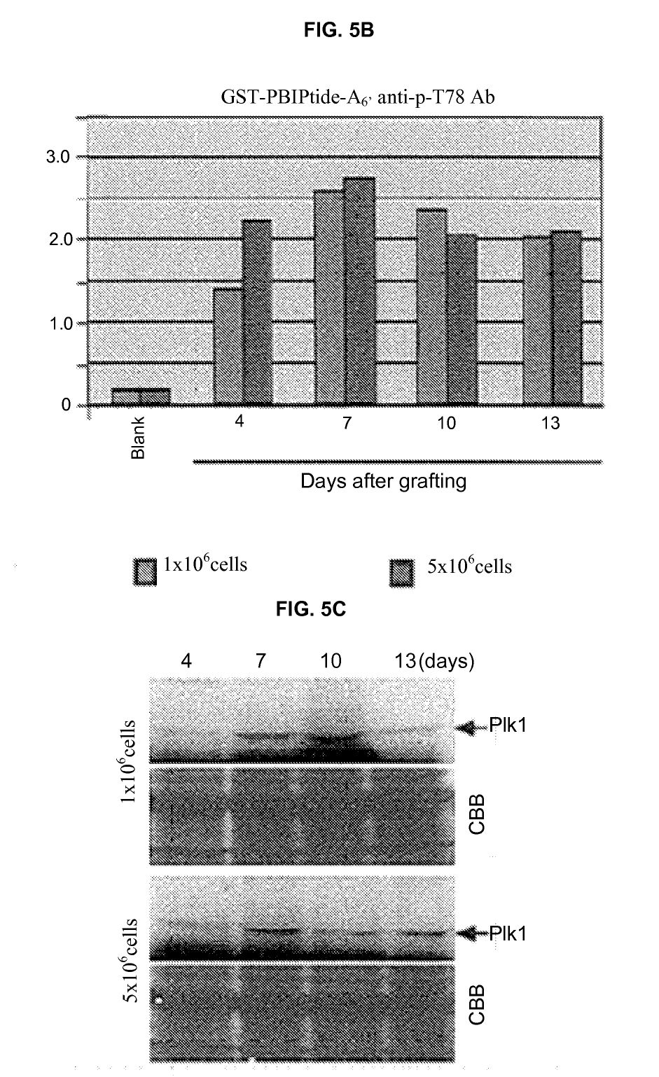

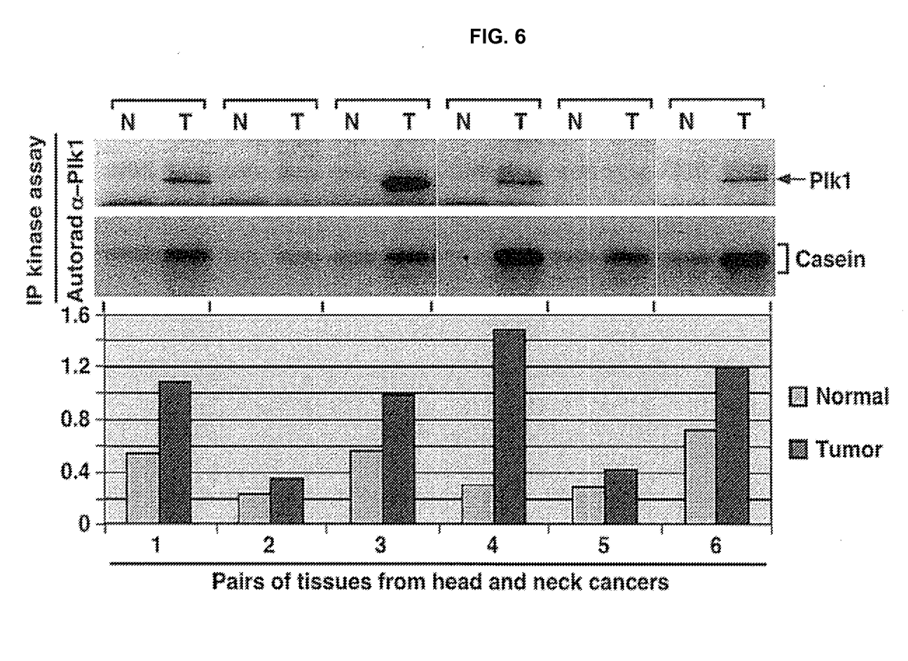

As shown in FIG. 5, athymic engrafted with B16 mouse tumor cells (over expressing Plk1) develop tumors in athymic mice engrafted. As shown in FIG. 5B Plk1 kinase activity was measured in total cellular protein extracted from tumors obtained from the mice shown in FIG. 5A. For a control, expression of Plk1 in the tumors obtained from the mice shown in FIG. 5A was determined by Western blots (see 5C). These results demonstrate that tumors can be screened for Plk1 kinase activity. As shown in FIG. 6, the kinase assay disclosed herein can also be used to test for kinase activity in human tumors.

For nude mouse xenografting, female athymic (NCr-nu/nu) mice were injected subcutaneously with the indicated numbers of B16 mouse tumor cells. At the indicated days after grafting, mice were killed and photographed. The resulting tumors were surgically removed, lysed in KC-plus buffer, and then subjected to either ELISA or immunoprecipitation kinase assay. Nude mice bearing xenografted B16 tumors were injected with BrdU (0.2 ml, 10 mM BrdU/100 g of body weight) 2 hours before termination. Tumors were collected and fixed in Formalin for 24 hours and then transferred into 70% ethanol. Fixed tumors were embedded in paraffin and sectioned with 5-μm intervals. Consecutive tumor sections were subjected to either anti-BrdU or H&E staining analyses. Photographs were taken using a Nikon microscope.

### Example 7

### Phosphorylation of GST-PBIPtide is Dependent on the Kinase Activity of Plk1

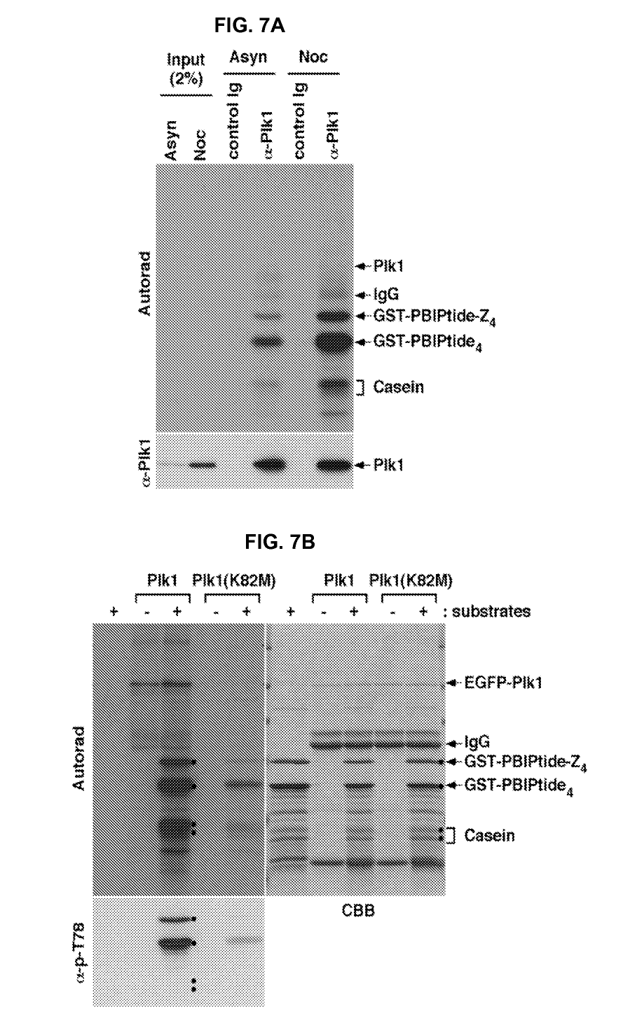

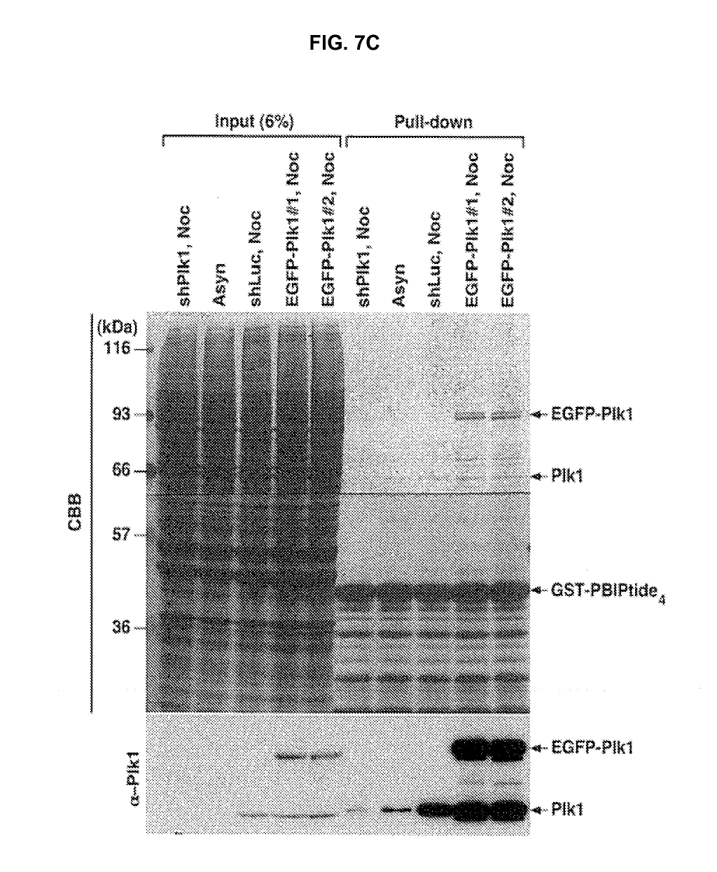

This example describes the determination that the phosphorylation of the GST-PBIPtide is dependent Plk1 kinase activity of Plk1. As shown in FIG. 7A-7C wild-type Plk1, but not the respective kinase-inactive form, efficiently phosphorylates GST-PBIPtide. Endogenous Plk1 immunoprecipitated from either asynchronously growing (Asyn) or nocodazole-treated (Noc) HeLa cells were subjected to kinase reactions in the presence of [γ-³²P]-ATP (see FIG. 7A). Both GST-PBIPtides (GST-PBIPtide-Z₄ and GST-PBIPtide₄) and an in vitro Plk1 phospho-transfer target, casein, were used as substrates in a single reaction tube. Samples were separated by SDS-PAGE, measured by autoradiography, and then immunoblotted with anti-Plk1 antibody (see FIG. 7A). As shown in FIG. 7B, HeLa cells expressing either EGFP-Plk1 or the corresponding kinase-inactive Plk1 (K82M) were subjected to anti-Plk1 immunoprecipitation. The immunoprecipitates were then subjected to kinase reactions as the samples shown in FIG. 7A. Samples were separated by SDS-PAGE, measured by autoradiography, and then blotted with anti-phospho-T78 antibody to examine the level of the phospho-T78 epitope generated. Later, the same membrane was stained with Coomassie (CBB). Dots indicate the positions of each substrate. As shown in FIG. 7C, HeLa cells infected with either shPlk1- or control shLuc-encoding lentivirus, or two independent HeLa cultures infected with adenovirus expressing EGFP-Plk1 were all treated with nocodazole for 16 hours to arrest the cells in prometaphase. Asynchronously growing HeLa cells (Asyn) were also included as a comparison. Total cellular lysates were prepared in TBSN buffer containing phosphatase inhibitors, and then incubated with the indicated GST-PBIPtide immobilized to the beads. The precipitated samples were separated by SDS-PAGE, immunoblotted with anti-Plk1 antibody to detect the co-precipitated Plk1, and then stained with Coomassie (CBB). Note that precipitation of GST-PBIPtide₄ co-precipitates a Coomassie-stainable level of transfected EGFP-Plk1 and endogenous Plk1. These results show that it is the kinase activity of Plk1 that is responsible for the phosphorylation of the GST-PBIPtide.

### Example 8

### Precipitation of Plk1 and Plx1 Using GST-PBIPtide

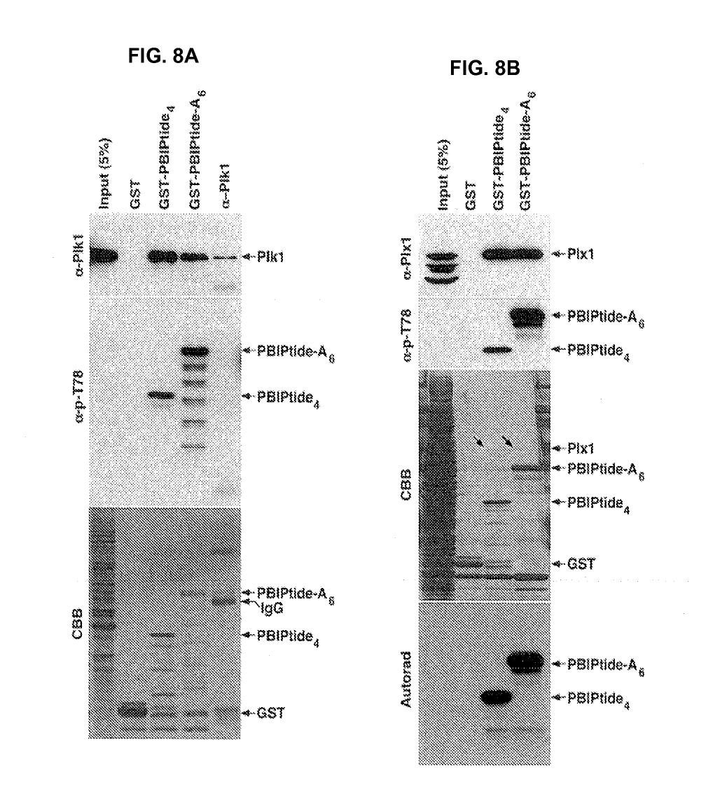

GST-PBIPtide efficiently precipitates Plk1 and its Xenopus laevis homolog, Plx1, from total cell lysates. As shown in FIG. 8A, mitotic HeLa lysates were prepared in TBSN containing phosphatase inhibitors and incubated with bead-bound forms of control GST or the indicated GST-PBIPtides. Anti-Plk1 immunoprecipitation was carried out as a comparison. Precipitates were separated and then immunoblotted with the indicated antibodies. Afterward, the same membrane was stained with Coomassie (CBB). As shown in FIG. 8B, cytostatic factor (CSF)-arrested egg extracts from Xenopus laevis were diluted in TBSN buffer containing phosphatase inhibitors, and then incubated with the indicated ligands immobilized to the beads. Precipitates were washed and then subjected to in vitro kinase reaction in the presence of [γ-³²P]-ATP. The resulting samples were separated, transferred to PVDF membrane, and measured for ³²P by autoradiography. Subsequently, the same membrane was immunoblotted with the indicated antibodies and stained with Coomassie (CBB). The arrows in FIG. 8B indicates a weakly detectable Plx1 precipitated by GST-PBIPtides.

### Example 9

### Evaluation of Plk1 Activity in Human Tissue Samples Using Plk1 ELISA Assay

This example describes the use of the disclosed Plk1 ELISA assay in detecting Plk1 activity in human tumor samples.

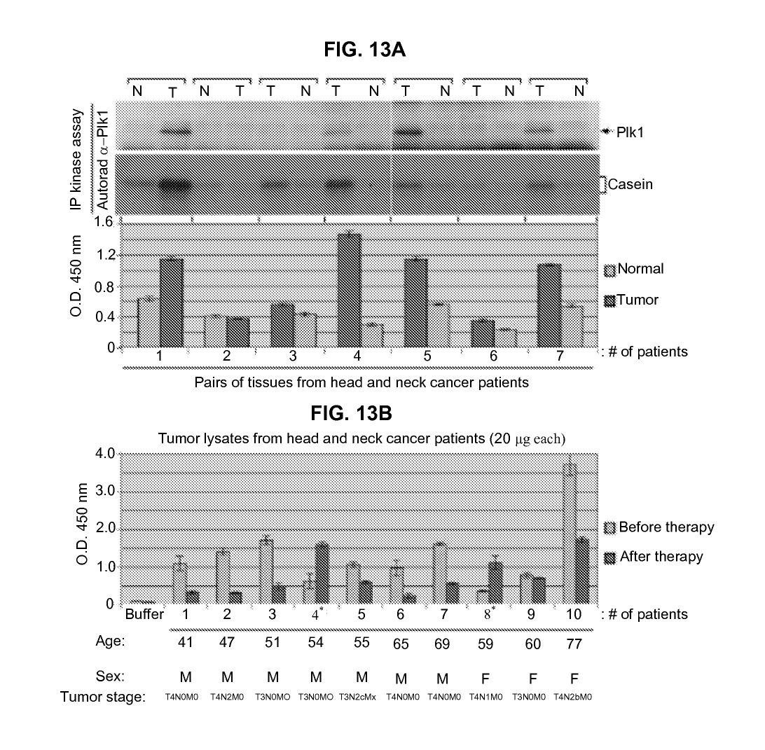

To determine whether the Plk1 ELISA assay can be used to quantify the Plk1 activity with human tissue samples, pairs of frozen tumors and its surrounding normal tissues were initially obtained from 7 Caucasian German head and neck cancer patients. Total lysates were prepared in KC-plus buffer and subjected to anti-Plk1 immunocomplex kinase assays with 2 mg of lysates using casein as substrate (FIG. 13A, top) and Plk1 ELISA assays with 2 mg of lysates using GST-PBIPtide-A6 as substrate (FIG. 13A, bottom). Note that the order of sample loading for patient #1 and #2 is different from the rest of the samples. The casein phosphorylation activity for the tumor sample from patient #1 is significantly higher than the ELISA-based Plk1 activity, raising the possibility that a contaminating kinase(s) co-precipitated with Plk1 immunoprecipitates could have contributed to the phosphorylation level of the generic substrate casein in vitro. Attempts to detect endogenous Plk1 by immunoblotting analysis with the total lysates failed due to a low percentage of mitotic cells in tissue samples. As shown in FIG. 13B, tumor biopsies were collected from Caucasian European head and neck cancer patients, who were placed under three rounds of a 3-week cycle chemotherapy regimen with Docetaxel (75 mg/m2), Cisplatinum (100 mg/m2), and 5-Fluorouracil (1000 mg/m2). All the biopsy samples collected before and after the three rounds of chemotherapy (a total of 10 biopsy pairs) were examined to determine the effect of the therapy on Plk1 activity. The samples before chemotherapy are needle biopsies, whereas the samples after chemotherapy are biopsies prepared immediately after surgical removal of the primary tumors. All the samples were inspected by two independent pathologists. Plk1 ELISA assays were carried out twice as in FIG. 13A with 20 μg of total lysates prepared from the respective tumors independently. Evaluation of tumor stages was conducted within 7 days before chemotherapy by carrying out magnetic resonance imaging or computed tomography of the head and neck tumors. Staging was based on the size of the primary tumor (T), the degree of regional lymph node involvement (N), and the absence or presence of distant metastases (M). Although the levels of Plk1 activity varied from one patient to another, this assay did indicate that seven of the ten pairs of samples exhibited significantly decreased Plk1 activity after the chemotherapeutic treatment. Interestingly, the two patients (marked with asterisks), who were delinquent in their scheduled second round (#8 patient) or third round (#4 patient) of treatment, exhibited high levels of Plk1 activity even after therapy. This could be due to reproliferation of tumor cells following their recovery from an incomplete chemotherapeutic treatment.

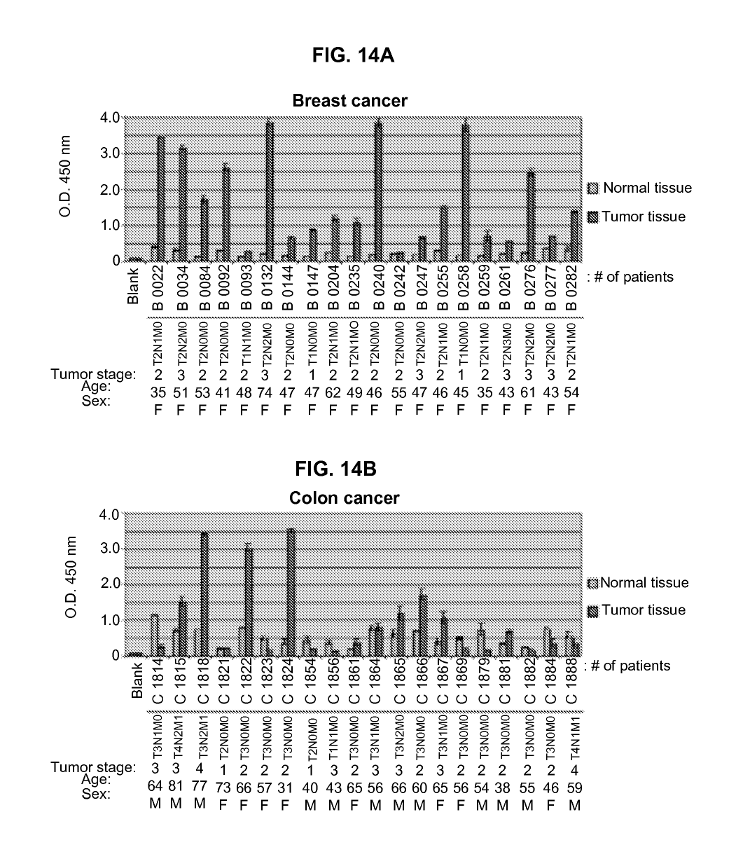

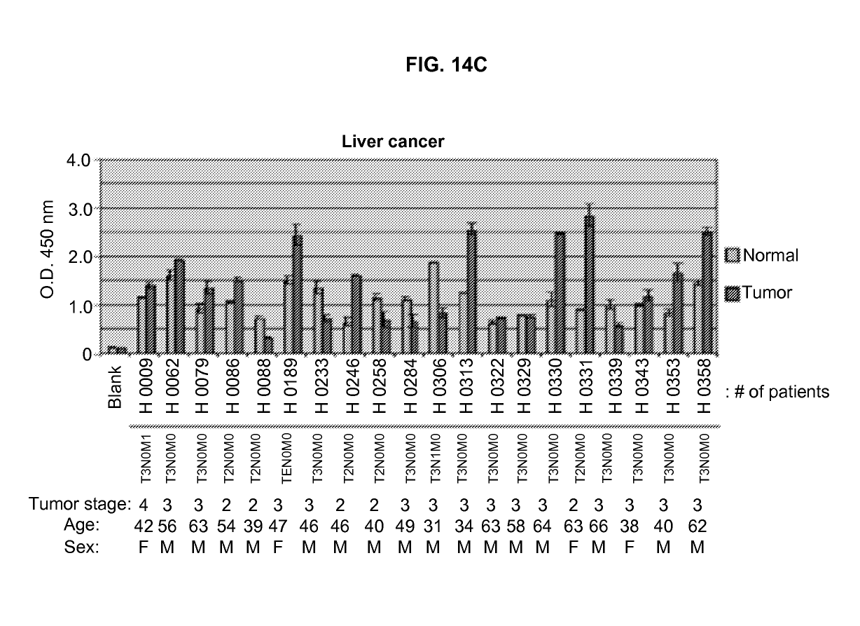

As shown in FIG. 14A-14C, biopsy samples of frozen tumor and the respective surrounding normal tissues were collected from a South Korean population suffering from breast, colon, or liver cancers (20 pairs per each cancer type). Samples were subjected to Plk1 ELISA assays. Tumor staging was determined similarly as in FIG. 13. Notably, most of the breast cancer tissues exhibited elevated levels of Plk1 activity in comparison to the corresponding normal tissues. However, only a fraction of colon and liver cancer tissues displayed elevated Plk1 activities in cancer tissues. These findings suggest that upregulated Plk1 activity could serve as an important prognostic indicator that foretells the predisposition for breast cancer development or the aggressiveness of breast cancer tumorigenesis.

### Example 10

### Identification of Plk1 Inhibitors

This example describes the methods that can be used to identify agents that inhibit Plk1 kinase activity.

A library of chemical compounds is obtained, for example from the Developmental Therapeutics Program NCl/NIH, and screened for their effect on the kinase activity using the assays disclosed herein and exemplified by Examples 1-9. Samples containing Plk1 (for example isolated Plk1 or cell lysates containing Plk1) are contacted in multiwell plates with one or more test agents in serial dilution, for example 1 nM to 1 mM of test agent. The sample is then contacted to multiwell plates that contain one of the peptide substrates of Plk1 disclosed herein for a period of time sufficient for the Plk1 to phosphorylate the peptide when the Plk1 kinase in not inhibited. In some examples, the peptide is attached to the plate, for example through a GST moiety. The degree of phosphorylation of the peptides present in the well is determined, for example by contacting the wells containing the peptides with a specific binding agent that only binds to the peptide when phosphorylated, for example using an antibody that specifically binds to the phospho-peptide or the phospho-peptide-bound Plk1. Agents identified as inhibitors of Plk1 kinase activity, for example be virtue of reducing the phosphorylation of the peptide in a dose dependent manner, are used as lead compounds to identify other agents having even greater inhibitory effects on Plk1 kinase activity. For example, chemical analogs of identified chemical entities, or variant, fragments of fusions of peptide agents, are tested for their activity methods described herein. Candidate agents also can be tested in cell lines and animal models to determine their therapeutic value. The agents also can be tested for safety in animals, and then used for clinical trials in animals or humans.

### Example 11

### Effect of Plk1 Inhibitors on Tumor Growth in Athymic Xenograft Assays

Inhibitors of Plk1 function identified using the methods disclosed herein (see for example Example 10) are tested in vivo to determine anti-tumor activity using nude mouse xenograft model systems. Female athymic (NCr-nu/nu) mice are injected subcutaneously with various human cancer cells (HeLa, Bel7402, MCF7, MIA PaCa, B 16, etc.) and the resulting tumors are treated with compounds identified with the assays disclosed herein. Tumor size and total body weights are measured every three days. To further verify the activity of these Plk1 inhibitors, the tumor masses from the control and experimental animals are removed to prepare tumor extracts and analyzed for Plk1 activity with the disclosed assays. Direct immunoblot analysis are performed to reveal whether tumor extracts from both control and Plk1 inhibitor-treated animals have similar steady-state levels of Plk1. Whether the above tested Plk1 inhibitors possess preclinical evidence of anti-tumor activity and low toxicity in animal tumor models, either as a single agent or in synergistic combination with other chemotherapeutic agents, is also examined. While this disclosure has been described with an emphasis upon particular embodiments, it will be obvious to those of ordinary skill in the art that variations of the particular embodiments may be used, and it is intended that the disclosure may be practiced otherwise than as specifically described herein. Features, characteristics, compounds, chemical moieties, or examples described in conjunction with a particular aspect, embodiment, or example of the invention are to be understood to be applicable to any other aspect, embodiment, or example of the invention. Accordingly, this disclosure includes all modifications encompassed within the spirit and scope of the disclosure as defined by the following claims.

1. An isolated peptide comprising two to ten consecutive repeats of the amino acid sequence set forth as X₁X₂AX₃X₄X₅PLHSTX₆X₇X₈X₉X₁₀X₁₁X₁₂ (SEQ ID NO: 1), in which within each repeat X₁ is independently any amino acid or no amino acid, X₂ is independently any amino acid or no amino acid, X₃ is independently any amino acid, X₄ is independently any amino acid, X₅ is independently any amino acid, X₆ is independently any amino acid, X₇ is independently any amino acid, X₈ is independently any amino acid or no amino acid, X₉ is independently any amino acid or no amino acid, X₁₀ is independently any amino acid or no amino acid, X₁₁ is independently any amino acid or no amino acid, and X₁₂ is independently any amino acid or no amino acid, and wherein the two to ten consecutive repeats of SEQ ID NO: 1 are joined a peptide linker between two and ten amino acids in length, wherein the peptide linker separates consecutive repeats of SEQ ID NO: 1.

2. The isolated peptide of claim 1, wherein X₁ is independently any amino acid or no amino acid, X₂ is independently any amino acid or no amino acid, X₃ is phenylalanine, X₄ is aspartic acid, X₅ is proline, X₆ is independently any amino acid, X₇ is independently any amino acid, X₈ is independently any amino acid or no amino acid, X₉ is independently any amino acid or no amino acid, X₁₀ is independently any amino acid or no amino acid, X₁₁ is independently any amino acid or no amino acid, and X₁₂ is independently any amino acid or no amino acid.

3. The isolated peptide of claim 1, wherein X₁ is independently any amino acid or no amino acid, X₂ is independently any amino acid or no amino acid, X₃ is phenylalanine, X₄ is aspartic acid, X₅ is proline, X₆ is alanine, X₇ isoluecine, X₈ is independently any amino acid or no amino acid, X₉ is independently any amino acid or no amino acid, X₁₀ is independently any amino acid or no amino acid, X₁₁ is independently any amino acid or no amino acid, and X₁₂ is independently any amino acid or no amino acid.

4. The isolated peptide of claim 1, wherein X₁ is tyrosine, X₂ is glutamic acid, X₃ is phenylalanine, X₄ is aspartic acid, X₅ is proline, X₆ is alanine, X₇ is isoluecine, X₈ is tyrosine, X₉ is alanine, X₁₀ is aspartic acid, X₁₁ is glutamic acid, and X₁₂ is glutamic acid.

5. The isolated peptide of claim 1, wherein X₁ is phenylalanine, X₂ is glutamic acid, X₃ is phenylalanine, X₄ is aspartic acid, X₅ is proline, X₆ is alanine, X₇ is isoluecine, X₈ is phenylalanine, X₉ is alanine, X₁₀ is aspartic acid, X₁₁ is glutamic acid, and X₁₂ is glutamic acid.

6.-8. (canceled)

9. The isolated peptide of claim 1, wherein the peptide linker comprises the amino acid sequence set forth as GGPGG (SEQ ID NO: 12).

10. (canceled)

11. The isolated peptide of claim 1, wherein the peptide is detectably labeled.

12. An isolated nucleic acid comprising a nucleotide sequence encoding the peptide of claim 1.

13. A method for detecting Plk1 kinase activity in a biological sample, comprising:
contacting a biological sample with the peptide of claim 1 in the presence of adenosine triphosphate, or an analog thereof, for a period of time sufficient for Plk1 to phosphorylate the peptide; and
detecting phosphorylation of the peptide, wherein the peptide is phosphorylated on one or more threonine residues,
thereby detecting the kinase activity of Plk1 in a biological sample.

14. The method of claim 13, further comprising contacting the biological sample with a specific binding agent that specifically binds to the peptide when the peptide is phosphorylated on one or more threonine residues, wherein the specific binding agent does not specifically bind to the peptide when the peptide is not phosphorylated on one or more threonine residues, and wherein detecting a complex formed between the specific binding agent and the peptide detects the phosphorylated peptide.

15. The method of claim 13, further comprising comparing the amount of the phosphorylated peptide detected with a control.

16. The method of claim 15, wherein the control is a value indicative of the amount of phosphorylated peptide formed from basal phosphorylation of the peptide, or a value indicative of the amount of phosphorylated peptide formed in the presence of a known amount of isolated Plk1.

17. The method of claim 14, wherein the specific binding agent is isolated Plk1 or an antibody that specifically binds to peptide when the peptide is phosphorylated on a threonine residue.

18. (canceled)

19. The method of claim 14, wherein the specific binding agent is detectably labeled.

20. The method of claim 13, wherein the peptide is detectably labeled.

21. The method of claim 14, further comprising contacting the biological sample with an antibody that specifically binds to the specific binding agent.

22. The method of claim 21, wherein the antibody is detectably labeled.

23. The method of claim 13, wherein the peptide is immobilized on a solid support.

24. (canceled)

25. A method for detecting a cancer or determining a predisposition for developing a cancer a subject, the method comprising:
obtaining a biological sample from the subject;
contacting the sample with the peptide according to claim 1 in the presence of adenosine triphosphate, or an analog thereof, for a period of time sufficient for Plk1 to phosphorylate the peptide;
detecting an amount of phosphorylated peptide;
comparing the amount of phosphorylated peptide formed with a control, wherein an increase in the amount of phosphorylated peptide formed relative to the control indicates that the subject has cancer or a predisposition for developing cancer.

26. The method of claim 25, wherein the control is a value indicative of the amount of complex formed from basal phosphorylation of the peptide, or a value indicative of the amount of complex formed in the presence of a known amount of isolated Plk1 or the amount of complex formed in a sample not contacted with the test agent.

27. A method for monitoring a subjects response to treatment for cancer, the method comprising:
obtaining a first biological sample at a first time point and a second biological sample at second later time point from a subject being treated for cancer;
contacting the first biological sample with the peptide according to claim 1 in the presence of adenosine triphosphate, or an analog thereof, for a period of time sufficient for Plk1 to phosphorylate the peptide
detecting a first amount of phosphorylated peptide formed from contacting the peptide with the first biological sample;
contacting the second biological sample with the peptide according to claim 1 in the presence of adenosine triphosphate, or an analog thereof, for a period of time sufficient for Plk1 to phosphorylate the peptide;
detecting a second amount of phosphorylated peptide formed from contacting the peptide with the second biological sample; and
comparing the first amount of phosphorylated peptide formed with the second amount of phosphorylated peptide formed, wherein an increase in the second amount of phosphorylated peptide formed relative to the first amount of phosphorylated peptide formed indicates that the subject is not responding to the treatment for cancer and wherein a decrease in the second amount of phosphorylated peptide formed relative to the first amount of phosphorylated peptide formed indicates that the subject is responding to the treatment for cancer.

28. The method of claim 25, further comprising contacting the biological sample(s) with a specific binding agent that specifically binds to the peptide when the peptide is phosphorylated on one or more threonine residues or a specific binding agent that specifically binds to the peptide when the peptide is not phosphorylated on a threonine residue; and
detecting a complex formed between the peptide and the specific binding agent that specifically binds to the peptide when the peptide is phosphorylated on one or more threonine residues, wherein detecting a complex formed between the peptide and the specific binding agent that specifically binds to the peptide when the peptide is phosphorylated identifies the test agent as one that does not inhibit Plk1 kinase activity; or
detecting a complex formed between the peptide and the specific binding agent that specifically binds to the peptide when the peptide is not phosphorylated, wherein detecting a complex formed between the peptide and the specific binding agent that specifically binds to the peptide when the peptide is not phosphorylated identifies the test agent as one that inhibits Plk1 kinase activity.

29. The method of claim 28, wherein the specific binding agent is an antibody that specifically binds to the peptide when the peptide is phosphorylated on a threonine residue, isolated Plk1 or an antibody that specifically binds to the peptide when the peptide is not phosphorylated.

30.-31. (canceled)

32. The method of claim 28, wherein the specific binding agent is detectably labeled.

33. The method of claim 28, further comprising contacting the sample with an antibody that specifically binds to the specific binding agent.

34. The method of claim 33, wherein the antibody that specifically binds to the specific binding agent is detectably labeled.

35. The method of claim 25, wherein the peptide is immobilized on a solid support.

36.-45. (canceled)

46. A kit for detecting the activity of Plk1, the kit comprising one or more peptides according to claim 1.

47. The method of claim 27, further comprising contacting the biological sample(s) with a specific binding agent that specifically binds to the peptide when the peptide is phosphorylated on one or more threonine residues or a specific binding agent that specifically binds to the peptide when the peptide is not phosphorylated on a threonine residue; and
detecting a complex formed between the peptide and the specific binding agent that specifically binds to the peptide when the peptide is phosphorylated on one or more threonine residues, wherein detecting a complex formed between the peptide and the specific binding agent that specifically binds to the peptide when the peptide is phosphorylated and identifies the test agent as one that does not inhibit Plk1 kinase activity; or
detecting a complex formed between the peptide and the specific binding agent that specifically binds to the peptide when the peptide is not phosphorylated, wherein detecting a complex formed between the peptide and the specific binding agent that specifically binds to the peptide when the peptide is not phosphorylated identifies the test agent as one that inhibits Plk1 kinase activity.

48. The method of claim 47, wherein the specific binding agent is an antibody that specifically binds to the peptide when the peptide is phosphorylated on a threonine residue, isolated Plk1, or an antibody that specifically binds to the peptide when the peptide is not phosphorylated.

49. The method of claim 47, wherein the specific binding agent is detectably labeled.

50. The method of claim 47, further comprising contacting the sample with an antibody that specifically binds to the specific binding agent.

51. The method of claim 52, wherein the antibody that specifically binds to the specific binding agent is detectably labeled.

52. The method of claim 47, wherein the peptide is immobilized on solid support.
